(19) United States
(12) Patent Application Publication
(10) Pub. No.: US 2021/0265009 A1
(43) Pub. Date: Aug. 26, 2021

(54) Artificial Intelligence-Based Base Calling of Index Sequences

(71) Applicant: Illumina, Inc., San Diego, CA (US)

(72) Inventors: Kishore JAGANATHAN, San Francisco, CA (US); Amirali KIA, San Mateo, CA (US)

(73) Assignee: Illumina, Inc., San Diego, CA (US)

(21) Appl. No.: 17/175,546

(22) Filed: Feb. 12, 2021

Related U.S. Application Data
(60) Provisional application No. 62/979,384, filed on Feb. 20, 2020.

(51) Int. Cl. G16B 25/10; G16B 40/10; G06N 3/08

(52) U.S. Cl. CPC G16B 25/10; G06N 3/08; G16B 40/10

(57) ABSTRACT

The technology disclosed relates to artificial intelligence-based base calling of index sequences. The technology disclosed accesses index images generated for the index sequences during index sequencing cycles of a sequencing run. The index images depict intensity emissions generated as a result of nucleotide incorporation in the index sequences during the sequencing run. The technology disclosed normalizes an index image from a current index sequencing cycle based on (i) intensity values of index images from one or more preceding index sequencing cycles, (ii) intensity values of index images from one or more succeeding index sequencing cycles, and (iii) intensity values of index images from the current index sequencing cycle. The technology disclosed processes normalized versions of the index images through a neural network-based base caller and generates a base call for each of the index sequencing cycles, thereby producing index reads for the index sequences.

### PRIORITY APPLICATION

This application claims priority to and benefit of U.S. Provisional Patent Application No. 62/979,384, titled “ARTIFICIAL INTELLIGENCE-BASED BASE CALLING OF INDEX SEQUENCES,” filed 20 Feb. 2020 (Attorney Docket No. ILLM 1015-1/IP-1857-PRV). The priority application is hereby incorporated by reference for all purposes as if fully set forth herein.

### FIELD OF THE TECHNOLOGY DISCLOSED

The technology disclosed relates to artificial intelligence type computers and digital data processing systems and corresponding data processing methods and products for emulation of intelligence (i.e., knowledge based systems, reasoning systems, and knowledge acquisition systems); and including systems for reasoning with uncertainty (e.g., fuzzy logic systems), adaptive systems, machine learning systems, and artificial neural networks. In particular, the technology disclosed relates to using deep neural networks such as deep convolutional neural networks for analyzing data.

### INCORPORATIONS

The following are incorporated by reference as if fully set forth herein:

U.S. Provisional Patent Application No. 62/979,414, titled “ARTIFICIAL INTELLIGENCE-BASED MANY-TO-MANY BASE CALLING,” filed 20 Feb. 2020 (Attorney Docket No. ILLM 1016-1/IP-1858-PRV);

U.S. Provisional Patent Application No. 62/979,385, titled “KNOWLEDGE DISTILLATION-BASED COMPRESSION OF ARTIFICIAL INTELLIGENCE-BASED BASE CALLER,” filed 20 Feb. 2020 (Attorney Docket No. ILLM 1017-1/IP-1859-PRV);

U.S. Provisional Patent Application No. 63/072,032, titled “DETECTING AND FILTERING CLUSTERS BASED ON ARTIFICIAL INTELLIGENCE-PREDICTED BASE CALLS,” filed 28 Aug. 2020 (Attorney Docket No. ILLM 1018-1/IP-1860-PRV);

U.S. Provisional Patent Application No. 62/979,412, titled “MULTI-CYCLE CLUSTER BASED REAL TIME ANALYSIS SYSTEM,” filed 20 Feb. 2020 (Attorney Docket No. ILLM 1020-1/IP-1866-PRV);

U.S. Provisional Patent Application No. 62/979,411, titled “DATA COMPRESSION FOR ARTIFICIAL INTELLIGENCE-BASED BASE CALLING,” filed 20 Feb. 2020 (Attorney Docket No. ILLM 1029-1/IP-1964-PRV);

U.S. Provisional Patent Application No. 62/979,399, titled “SQUEEZING LAYER FOR ARTIFICIAL INTELLIGENCE-BASED BASE CALLING,” filed 20 Feb. 2020 (Attorney Docket No. ILLM 1030-1/IP-1982-PRV);

U.S. Nonprovisional patent application Ser. No. 16/825,987, titled “TRAINING DATA GENERATION FOR ARTIFICIAL INTELLIGENCE-BASED SEQUENCING,” filed 20 Mar. 2020 (Attorney Docket No. ILLM 1008-16/IP-1693-US);

U.S. Nonprovisional patent application Ser. No. 16/825,991 titled “ARTIFICIAL INTELLIGENCE-BASED GENERATION OF SEQUENCING METADATA,” filed 20 Mar. 2020 (Attorney Docket No. ILLM 1008-17/IP-1741-US);

U.S. Nonprovisional patent application Ser. No. 16/826,126, titled “ARTIFICIAL INTELLIGENCE-BASED BASE CALLING,” filed 20 Mar. 2020 (Attorney Docket No. ILLM 1008-18/IP-1744-US);

U.S. Nonprovisional patent application Ser. No. 16/826,134, titled “ARTIFICIAL INTELLIGENCE-BASED QUALITY SCORING,” filed 20 Mar. 2020 (Attorney Docket No. ILLM 1008-19/IP-1747-US); and

U.S. Nonprovisional patent application Ser. No. 16/826,168, titled “ARTIFICIAL INTELLIGENCE-BASED SEQUENCING,” filed 21 Mar. 2020 (Attorney Docket No. ILLM 1008-20/IP-1752-PRV-US).

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

Improvements in Next-Generation Sequencing (NGS) technology have greatly increased sequencing speed and data output, resulting in the massive sample throughput of current sequencing platforms. Approximately ten years ago, the Illumina Genome Analyzer™ was capable of generating up to one gigabyte of sequence data per run. Today, the Illumina NovaSeg™ series of systems are capable of generating up to two terabytes of data in two days, which represents a greater than 2000× increase in capacity.

A key to utilizing this increased capacity is multiplexing, which enables pooling and sequencing of multiple libraries simultaneously during a single sequencing run through addition of unique index sequence (“barcode”) to each DNA fragment during library preparation. Sequencing reads are sorted to their respective samples during demultiplexing, allowing for proper alignment.

An opportunity arises to use artificial intelligence and neural networks for base calling index sequences. Higher base calling throughput and increased base calling accuracy may result.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The color drawings also may be available in PAIR via the Supplemental Content tab.

In the drawings, like reference characters generally refer to like parts throughout the different views. Also, the drawings are not necessarily to scale, with an emphasis instead generally being placed upon illustrating the principles of the technology disclosed. In the following description, various implementations of the technology disclosed are described with reference to the following drawings, in which:

FIG. 1 shows one implementation of sequencing of polynucleotides from indexed libraries.

FIG. 2 shows one implementation of sequencing a target sequence to generate a target read and sequencing an index sequence to generate an index read.

FIG. 3 illustrates one implementation of normalizing index images.

FIG. 4 depicts one implementation of processing normalized index images through the neural network-based base caller for base calling.

FIG. 5 shows one implementation of expanding the normalization of index images to non-current index sequencing cycles.

FIG. 6 illustrates one implementation of normalizing index images using at least one index image that depicts one or more nucleotides in the detectable signal state.

FIG. 7 depicts one implementation of base calling target sequences and index sequences.

FIG. 8 illustrates one implementation of preprocessing that uses augmentation.

FIGS. 9 and 10 depict pixel intensity histograms of red and green images of two target sequencing cycles (cycles 1 and 151) of a first target read (Read 1).

FIGS. 11, 12, 13, 14, 15, 16, 17, and 18 depict pixel intensity histograms of red and green images of eight index sequencing cycles (cycles 152, 153, 154, 155, 156, 157, 158, and 159) of a first index read (Index Read 1).

FIGS. 19, 20, 21, 22, 23, 24, 25, and 26 depict pixel intensity histograms of red and green images of eight index sequencing cycles (cycles 160, 161, 162, 163, 164, 165, 166, and 167) of a second index read (Index Read 2).

FIGS. 27 and 28 depict pixel intensity histograms of red and green images of two target sequencing cycles (cycles 168 and 169) of a second target read (Read 2).

FIG. 29 shows that for a sequencing run that uses four index sequences for multiplexing four samples, the index base calling performance of the neural network-based base caller drops when the index images are not normalized.

FIG. 30 shows that for a sequencing run that uses two index sequences for multiplexing two samples, the index base calling performance of the neural network-based base caller drops when the index images are not normalized.

FIG. 31 shows that for a sequencing run that uses a single index sequence for sequencing a single sample, the index base calling performance of the neural network-based base caller drops when the index images are not normalized.

FIG. 32 is a computer system that can be used to implement the technology disclosed.

FIG. 33 depicts another implementation of base calling target sequences and index sequences.

FIG. 34 is one implementation of a flow chart of an artificial intelligence-based method of base calling analytes at index sequencing cycles of a sequencing run.

FIG. 35 is one implementation of a flow chart of an artificial intelligence-based method of base calling target sequences and index sequences.

### DETAILED DESCRIPTION

The following discussion is presented to enable any person skilled in the art to make and use the technology disclosed and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown but is to be accorded the widest scope consistent with the principles and features disclosed herein.

### Multiplexing

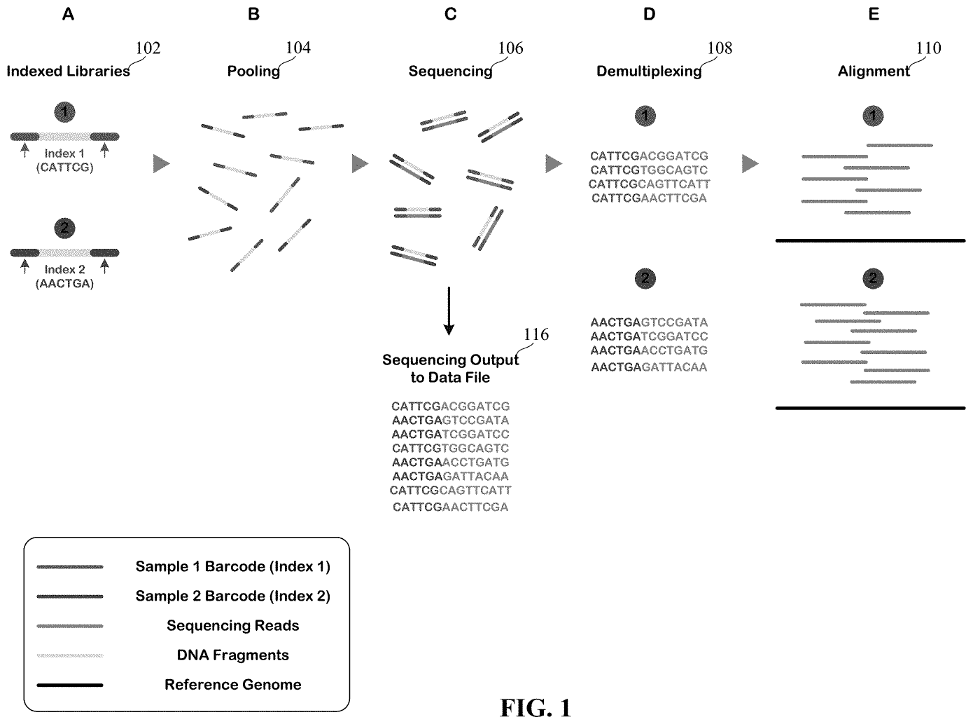

FIG. 1 shows one implementation of sequencing of polynucleotides from indexed libraries. When polynucleotides from different libraries are pooled or multiplexed for sequencing, the polynucleotides from each library are modified to include a library-specific index sequence. During sequencing, the index sequences are sequenced along with target polynucleotide sequences from the libraries. An index sequence is associated with a target polynucleotide sequence so that the library from which the target sequence originated can be identified.

Additional details about multiplexing, index sequences, and demultiplexing can be found in Illumina, “Indexed Sequencing Overview Guide”, Document No. 15057455, v. 5, March 2019 and in Illumina's patent application publications US 2018/0305751, US 2018/0334712, US 2016/0110498, US 2018/0334711, and WO 2019/090251, each of which is incorporated herein by reference.

Panel A shows indexed libraries 102. Here, unique index sequences (“indexes”) are added to two different libraries during library preparation. The first index sequence (Index 1) has a barcode of “CATTCG.” The second index sequence (Index 2) has a barcode of “AACTGA.”

Panel B shows pooling 104. Here, the indexed libraries 102 are pooled together and loaded into the same flow cell lane.

Panel C shows sequencing 106 and sequencing output 116. Here, the indexed libraries 102 are sequenced together during a single instrument run. All sequences are then exported to an output file 116. The output file 116 comprises sequence reads (in green) coupled to corresponding index reads (in blue and magenta).

Panel D shows demultiplexing 108. Here, a demultiplexing algorithm sorts the sequence reads into different files according to their indexes.

Panel E shows alignment 110. Here, each set of the demultiplexed sequence reads is aligned to the appropriate reference sequence.

Target Sequences and Index Sequences

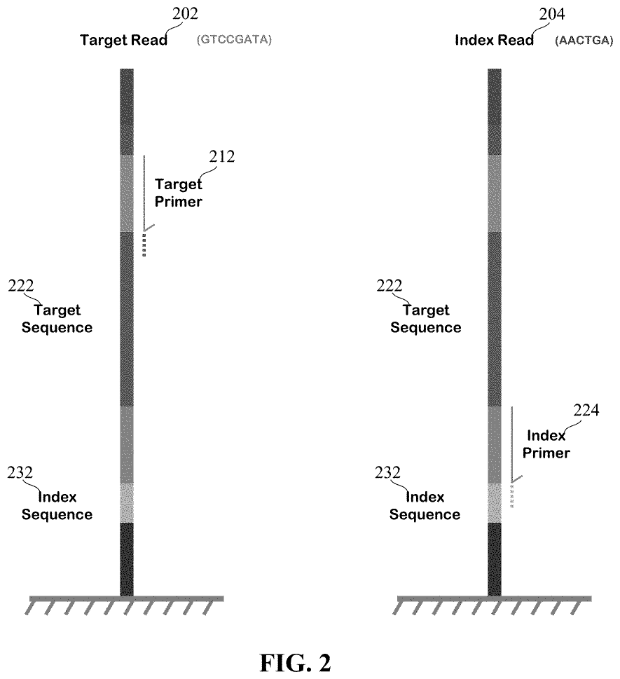

FIG. 2 shows one implementation of sequencing a target sequence 222 to generate a target read 202 (“GTCCGATA”) and sequencing an index sequence 232 to generate an index read 204 (“AACTGA”). The index sequence 232 can be a synthetic sequence of nucleotides that is coupled to the target sequence 222 during the template preparation step. The target sequence 222 can be naturally occurring DNA, RNA, or some other biological molecule. The length of the index sequence 232 can range from two to twenty nucleotides. For example, the index sequence 232 can be one to ten nucleotides long or four to six nucleotides long. A four-nucleotide index sequence gives the possibility of multiplexing 256 samples on the same array. A six-nucleotide index sequence enables 4096 samples to be processed on the same array.

During the sequencing 106, a target primer 212 traverses the target sequence 222 and produces the target read 202 (“GTCCGATA”) and an index primer 224 traverses the index sequence 232 and produces the index read 204 (“AACTGA”). In some implementations, the sequencing 106 is Illumina's single-indexed sequencing. In other implementations, the sequencing 106 is Illumina's dual-indexed sequencing.

Base calling is the process of determining the nucleotide composition of the target sequence 222 and the index sequence 232, i.e., the process of generating the target read 202 (“GTCCGATA”) and the index read 204 (“AACTGA”). Base calling involves analyzing image data, i.e., sequencing images produced during the sequencing 106 by a sequencing instrument such as Illumina's iSeq, HiSeqX, HiSeq 3000, HiSeq 4000, HiSeq 2500, NovaSeq 6000, NextSeq, NextSeqDx, MiSeq and MiSeqDx. The following discussion outlines how the sequencing images are generated and what they depict, in accordance with one implementation.

Base calling decodes the raw signal of the sequencing instrument, i.e., intensity data extracted from the sequencing images, into nucleotide sequences. In one implementation, the Illumina platforms employ Cyclic Reversible Termination (CRT) chemistry for base calling. The process relies on growing nascent strands complementary to template strands with fluorescently-labeled nucleotides, while tracking the emitted signal of each newly added nucleotide. The fluorescently-labeled nucleotides have a 3′ removable block that anchors a fluorophore signal of the nucleotide type.

Sequencing 106 occurs in repetitive cycles, each comprising three steps: (a) extension of a nascent strand (e.g., the target sequence 222, the index sequence 232) by adding the fluorescently-labeled nucleotide; (b) excitation of the fluorophore using one or more lasers of an optical system of the sequencing instrument and imaging through different filters of the optical system, yielding the sequencing images; and (c) cleavage of the fluorophore and removal of the 3′ block in preparation for the next sequencing cycle. Incorporation and imaging cycles are repeated up to a designated number of sequencing cycles, defining the read length. Using this approach, each cycle interrogates a new position along the template strands.

The tremendous power of the Illumina platforms stems from their ability to simultaneously execute and sense millions or even billions of analytes (e.g., clusters) undergoing CRT reactions. A cluster comprises approximately one thousand identical copies of a template strand, though clusters vary in size and shape. The clusters are grown from the template strand, prior to the sequencing run, by bridge amplification of the input library. The purpose of the amplification and cluster growth is to increase the intensity of the emitted signal since the imaging device cannot reliably sense fluorophore signal of a single strand. However, the physical distance of the strands within a cluster is small, so the imaging device perceives the cluster of strands as a single spot.

Sequencing 106 occurs in a flow cell—a small glass slide that holds the input strands. The flow cell is connected to the optical system, which comprises microscopic imaging, excitation lasers, and fluorescence filters. The flow cell comprises multiple chambers called lanes. The lanes are physically separated from each other and may contain different tagged sequencing libraries, distinguishable without sample cross contamination. The imaging device of the sequencing instrument (e.g., a solid-state imager such as a Charge-Coupled Device (CCD) or a Complementary Metal-Oxide-Semiconductor (CMOS) sensor) takes snapshots at multiple locations along the lanes in a series of non-overlapping regions called tiles. For example, there are hundred tiles per lane in Illumina's Genome Analyzer II and sixty-eight tiles per lane in Illumina's HiSeq 2000. A tile holds hundreds of thousands to millions of clusters.

The output of the sequencing 106 is the sequencing images, each depicting intensity emissions of the clusters and their surrounding background. Those sequencing cycles of the sequencing 106 that sequence the target sequence 222 are called “target sequencing cycles” and those sequencing cycles of the sequencing 106 that sequence the index sequence 232 are called “index sequencing cycles.” The sequencing images generated during the target sequencing cycles are called “target images” and the sequencing images generated during the index sequencing cycles are called “index images.”

The target images depict intensity emissions generated as a result of nucleotide incorporation in the target sequences during the sequencing 106. The index images depict intensity emissions generated as a result of nucleotide incorporation in the index sequences during the sequencing 106. The intensity emissions are from associated analytes and their surrounding background.

### Neural Network-Based Base Calling

The discussion now turns to the neural network-based base calling in which a neural network, i.e., a neural network-based base caller 430, is trained to map sequencing images to base calls 432.

The following discussion is organized as follows. First, the input to the neural network-based base caller 430 is described, in accordance with one implementation. Then, examples of the structure and form of the neural network-based base caller 430 are provided. Finally, the output of the neural network-based base caller 430 is described, in accordance with one implementation.

Additional details about the neural network-based base caller 430 can be found in U.S. Provisional Patent Application No. 62/821,766, titled “ARTIFICIAL INTELLIGENCE-BASED SEQUENCING,” (Attorney Docket No. ILLM 1008-9/IP-1752-PRV), filed on Mar. 21, 2019, which is incorporated herein by reference.

In one implementation, image patches are extracted from the target images and the index images. The extracted image patches are provided to the neural network-based base caller 430 as “input image data” for base calling. The image patches have dimensions w×h, where w (width) and h (height) are any numbers ranging from 1 and 10,000 (e.g., 3×3, 5×5, 7×7, 10×10, 15×15, 25×25). In some implementations, w and h are the same. In other implementations, w and h are different.

Sequencing 106 produces m image(s) per sequencing cycle for corresponding m image channels. In one implementation, each image channel corresponds to one of a plurality of filter wavelength bands. In another implementation, each image channel corresponds to one of a plurality of imaging events at a sequencing cycle. In yet another implementation, each image channel corresponds to a combination of illumination with a specific laser and imaging through a specific optical filter.

An image patch is extracted from each of the m image(s) to prepare the input image data for a particular sequencing cycle. In different implementations such as 4-, 2-, and 1-channel chemistries, m is 4 or 2. In other implementations, m is 1, 3, or greater than 4. The input image data is in the optical, pixel domain in some implementations, and in the upsampled, subpixel domain in other implementations.

Consider, for example, that sequencing 106 uses two different image channels: a red channel and a green channel. Then, at each sequencing cycle, sequencing 106 produces a red image and a green image. This way, for a series of k sequencing cycle, a sequence with k pairs of red and green images is produced as output.

The input image data comprises a sequence of per-cycle image patches generated for a series of k sequencing cycles of a sequencing run. The per-cycle image patches contain intensity data for associated analytes and their surrounding background in one or more image channels (e.g., a red channel and a green channel). In one implementation, when a single target analyte (e.g., cluster) is to be base called, the per-cycle image patches are centered at a center pixel that contains intensity data for a target associated analyte and non-center pixels in the per-cycle image patches contain intensity data for associated analytes adjacent to the target associated analyte.

The input image data comprises data for multiple sequencing cycles (e.g., a current sequencing cycle, one or more preceding sequencing cycles, and one or more successive sequencing cycles). In one implementation, the input image data comprises data for three sequencing cycles, such that data for a current (time t) sequencing cycle to be base called is accompanied with (i) data for a left flanking/context/previous/preceding/prior (time t−1) sequencing cycle and (ii) data for a right flanking/context/next/successive/subsequent (time t+1) sequencing cycle. In other implementations, the input image data comprises data for a single sequencing cycle. In yet other implementations, the input image data comprises data for 58, 75, 92, 130, 168, 175, 209, 225, 230, 275, 318, 325, 330, 525, or 625 sequencing cycles.

In one implementation, the neural network-based base caller 430 is a multilayer perceptron (MLP). In another implementation, the neural network-based base caller 430 is a feedforward neural network. In yet another implementation, the neural network-based base caller 430 is a fully-connected neural network. In a further implementation, the neural network-based base caller 430 is a fully convolutional neural network. In yet further implementation, the neural network-based base caller 430 is a semantic segmentation neural network.

In one implementation, the neural network-based base caller 430 is a convolutional neural network (CNN) with a plurality of convolution layers. In another implementation, it is a recurrent neural network (RNN) such as a long short-term memory network (LSTM), bi-directional LSTM (Bi-LSTM), or a gated recurrent unit (GRU). In yet another implementation, it includes both a CNN and an RNN.

In yet other implementations, the neural network-based base caller 430 can use 1D convolutions, 2D convolutions, 3D convolutions, 4D convolutions, 5D convolutions, dilated or atrous convolutions, transpose convolutions, depthwise separable convolutions, pointwise convolutions, 1×1 convolutions, group convolutions, flattened convolutions, spatial and cross-channel convolutions, shuffled grouped convolutions, spatial separable convolutions, and deconvolutions. It can use one or more loss functions such as logistic regression/log loss, multi-class cross-entropy/softmax loss, binary cross-entropy loss, mean-squared error loss, L1 loss, L2 loss, smooth L1 loss, and Huber loss. It can use any parallelism, efficiency, and compression schemes such TFRecords, compressed encoding (e.g., PNG), sharding, parallel calls for map transformation, batching, prefetching, model parallelism, data parallelism, and synchronous/asynchronous SGD. It can include upsampling layers, downsampling layers, recurrent connections, gates and gated memory units (like an LSTM or GRU), residual blocks, residual connections, highway connections, skip connections, peephole connections, activation functions (e.g., non-linear transformation functions like rectifying linear unit (ReLU), leaky ReLU, exponential liner unit (ELU), sigmoid and hyperbolic tangent (tanh)), batch normalization layers, regularization layers, dropout, pooling layers (e.g., max or average pooling), global average pooling layers, and attention mechanisms.

In one implementation, the neural network-based base caller 430 outputs a base call for a single target analyte for a particular sequencing cycle. In another implementation, it outputs a base call for each target analyte in a plurality of target analytes for the particular sequencing cycle. In yet another implementation, it outputs a base call for each target analyte in a plurality of target analytes for each sequencing cycle in a plurality of sequencing cycles, thereby producing a base call sequence for each target analyte.

### Preprocessing

In one implementation, image data from the target images and the index images is not directly fed as input to the neural network-based base caller 430. Instead, the target images and the index images are first preprocessed. However, the index images are preprocessed differently than the target images.

The base calling logic described herein accounts for the observation that index images depict nucleotides with low-complexity patterns in which some of the four bases A, C, T, and G are represented at a frequency of less than 15%, 10%, or 5% of all the nucleotides. This is the case because, for any given index sequencing cycle, an index image depicts intensity emissions of (1) multiple analytes that originate from the same sample and share the same index sequence, and also of (2) analytes that belong to different samples and have different index sequences.

The first type of analytes have the same index base for every index sequencing cycle. As a result, the index image ends up depicting the same nucleotide for multiple analytes. This reduces the nucleotide diversity of the index image.

The index image's nucleotide diversity is further reduced when the second type of analytes also end up having the same index base for certain index sequencing cycles. This happens for two reasons. First, the index sequences are short sequences with two to twenty index bases and thus do not have enough positions that can create significant mismatches between different index sequences. Second, often, up to only twenty samples are pooled for simultaneous sequencing. As a result, the number of different index sequences that can be depicted by an index image is not substantial. These factors lead to different index sequences having matching index bases at the same positions (base collision), which in turn causes the analytes with different index sequences to have the same index base for certain index sequencing cycles.

Low nucleotide diversity in the index images creates intensity patterns that lack signal diversity (contrast). On the other hand, the target images depict nucleotides with high-complexity patterns in which each of the four bases A, C, T, and G is represented at a frequency of at least 20%, 25%, or 30% of all the nucleotides. This is the case because the target sequences are often long (e.g., one-fifty bases) and are unique to each analyte regardless of the source sample. Therefore, unlike the index images, the target images have adequate signal diversity.

Convolution kernels and filters of the neural network-based base caller 430 are trained largely on the target images. So, when, during inference, the trained neural network-based base caller 430 is presented with index images that have not undergone preprocessing (raw index images), its base calling accuracy for the index reads drops because its convolution kernels and filters are trained to detect intensity patterns based on the contrast.

Bypassing preprocessing by training the neural network-based base caller 430 on large amounts of raw index images to introduce signal diversity is not feasible because only so many index sequences are published and made publicly available. Second, it is not uncommon for users to design custom index sequences and use them instead of the published index sequences. So, when trained on just the raw index images, the neural network-based base caller 430 does not generalize well during inference and is prone to overfitting.

One solution is to preprocess the index images using normalization. An index image from a current index sequencing cycle is normalized based on (i) intensity values of index images from one or more preceding index sequencing cycles, (ii) intensity values of index images from one or more succeeding index sequencing cycles, and (iii) intensity values of index images from the current index sequencing cycle.

Intensity values measure chemiluminescent signals produced due to nucleotide incorporations. Intensity values are encoded in “images” and represent “optical signals” that in turn contain “specific signals.” As used herein, the term “image” is intended to mean a representation of all or part of an object. The representation can be an optically detected reproduction. For example, an image can be obtained from fluorescent, luminescent, scatter, or absorption signals. The part of the object that is present in an image can be the surface or other xy plane of the object. An image is a 2-dimensional representation, but in some cases information in the image can be derived from 3 or more dimensions. An image need not include optically detected signals. Non-optical signals can be present instead (such as voltage, pH, or ion data). An image can be provided in a computer readable format or medium such as one or more of those set forth elsewhere herein. As used herein, the term “optical signal” is intended to include, for example, fluorescent, luminescent, scatter, or absorption signals. Optical signals can be detected in the ultraviolet (UV) range (about 200 to 390 nm), visible (VIS) range (about 391 to 770 nm), infrared (IR) range (about 0.771 to 25 microns), or other range of the electromagnetic spectrum. Optical signals can be detected in a way that excludes all or part of one or more of these ranges. As used herein, the term “specific signal” is intended to mean detected energy or coded information that is selectively observed over other energy or information such as background energy or information. For example, a specific signal can be an optical signal detected at a particular intensity, wavelength or color; an electrical signal detected at a particular frequency, power or field strength; or other signals known in the art pertaining to spectroscopy and analytical detection. In one implementation, the intensity values are extracted from two different color/intensity channel sequencing images. The identity of the four different nucleotide types/bases A, C, T, and G is encoded as a combination of the intensity values in the two color images, i.e., the first and second intensity channels. For example, a nucleic acid can be sequenced by providing a first nucleotide type (e.g., base T) that is detected in the first intensity channel, a second nucleotide type (e.g., base C) that is detected in the second intensity channel, a third nucleotide type (e.g., base A) that is detected in both the first and the second intensity channels, and a fourth nucleotide type (e.g., base G) that lacks a label that is not, or minimally, detected in either intensity channels. In some implementations, four intensity distributions (e.g., Gaussian distributions) are iteratively fitted to the intensity values in the first and the second intensity channels. The four intensity distributions correspond to the four bases A, C, T, and G. The intensity values in the first intensity channel are plotted against the intensity values in the second intensity channel (e.g., as a scatterplot), and the intensity values segregate into the four intensity distributions.

The normalization across index sequencing cycles also includes normalization across image channels within image data of the index sequencing cycles. For example, consider three index sequencing cycles: a first index sequencing cycle, a second index sequencing cycle, and a third index sequencing cycle. Also consider that each of the first, second, and third index sequencing cycles has two index images: a first index image (e.g., red index image) in a first image channel (e.g., red channel) and a second index image (e.g., green index image) in a second image channel (e.g., green channel). A red index image from the second index sequencing cycle is normalized based on (i) intensity values of red and green images from the first index sequencing cycle, (ii) intensity values of red and green images from the third index sequencing cycle, and (iii) intensity values of red and green images from the second index sequencing cycle. A green index image from the second index sequencing cycle is normalized based on (i) intensity values of red and green images from the first index sequencing cycle, (ii) intensity values of red and green images from the third index sequencing cycle, and (iii) intensity values of red and green images from the second index sequencing cycle.

The normalization includes index images from flanking index sequencing cycles because taken together, nucleotides depicted by the index images from the current, preceding, and succeeding index sequencing cycles are cumulatively more diverse than nucleotides depicted only by the index images from the current index sequencing cycle. Expanding the normalization to index images from the flanking index sequencing cycles also includes at least one index image from the preceding and/or succeeding index sequencing cycles that depicts one or more nucleotides in a detectable signal state. More details follow.

### Normalization of Index Images

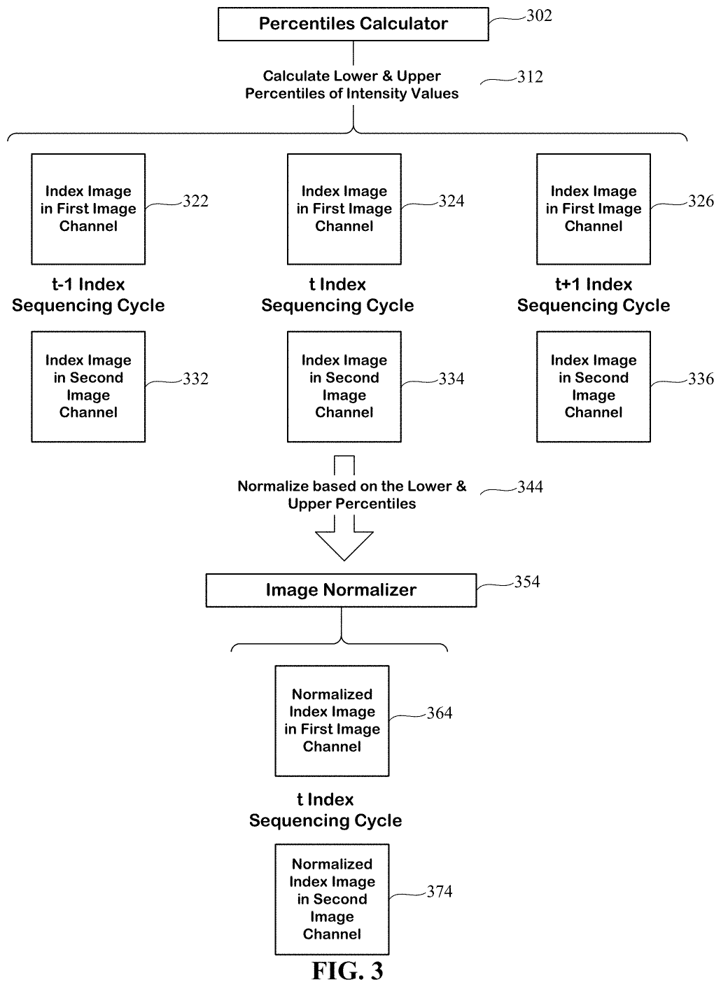

FIG. 3 illustrates one implementation of normalizing 344 index images.

A percentiles calculator 302 calculates 312 a lower percentile of (i) the intensity values of the index images 322, 332 from the preceding (time t−1) index sequencing cycle, (ii) the intensity values of the index images 326, 336 from the succeeding (time t+1) index sequencing cycles, and (iii) the intensity values of the index images 324, 334 from the current (time t) index sequencing cycle.

The percentiles calculator 302 is configured with percentiles calculation logic to calculate the percentile intensity values for the images. The percentiles calculator 302 can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media).

As discussed above, each index sequencing cycle can have 2, 3, 4, or more index images. Thus, the intensity values of the index images in the respective index image set from each of the preceding (time t−1) index sequencing cycle, the succeeding (time t+1) index sequencing cycles, and the current (time t) index sequencing cycle are used to normalize the intensity values of the index images in the index image set from the current (time t) index sequencing cycle.

In the illustrated implementation, each index sequencing cycle has two index images, one in a first image channel (e.g., red channel) and another in a second image channel (e.g., green channel).

In preferred implementations, the normalization of an index image in a first image channel (e.g., red channel) uses index images in the first image channel and also one or more index images in other image channels (e.g., green channel).

In other implementations, the normalization of an index image in a particular image channel only uses index images in that particular image channel and does not use index images in a different image channel. For example, in such an implementation, the current, normalized index image in the first channel 364 is generated only from the intensity values of the preceding index image in the first channel 322 and the succeeding index image in the first channel 326. Similarly, the current, normalized index image in the second channel 374 is generated only from the intensity values of the preceding index image in the second channel 332 and the succeeding index image in the second channel 336.

The percentiles calculator 302 also calculates 312 an upper percentile of (i) the intensity values of the index images 322, 332 from the preceding (time t−1) index sequencing cycle, (ii) the intensity values of the index images 326, 336 from the succeeding (time t+1) index sequencing cycle, and (iii) the intensity values of the index images 324, 334 from the current (time t) index sequencing cycle.

Then, based on the lower and upper percentiles, an image normalizer 354 generates normalized versions 364, 374 of the index images 324, 334 such that a first percentage of normalized intensity values are below the lower percentile, a second percentage of the normalized intensity values are above the upper percentile, and a third percentage of the normalized intensity values are between the lower and upper percentiles.

In one example, the lower percentile can be fifth percentile and the upper percentile can be ninety-fifth percentile. The normalized intensity value for the fifth percentile can be zero and the normalized intensity value for the ninety-fifth percentile can be one. Accordingly, in the normalized versions 364, 374 of the index images 324, 334, (i) five percent of the normalized intensity values are below zero, (ii) another five percent of the normalized intensity values are greater than one, and (iii) the remaining ninety percent of the normalized intensity values are between zero and one. The intensity values can be pixel intensity values, subpixel intensity values, or superpixel intensity values.

The normalization function can be mathematically expressed as:

$$normalized intensity value = \frac{intensity value - lower percentile}{upper percentile - lower percentile}$$

Thus, in one example, when the intensity value is that of the ninety-fifth percentile, the normalized intensity value is one, and when the intensity value is that of the fifth percentile, the normalized intensity value is zero.

In other implementations, the lower percentile can be tenth percentile and the upper percentile can be ninetieth percentile. In yet other implementations, the lower percentile can be any number between one and hundred, and the upper percentile is 100-the lower percentile. The normalized intensity values assigned to the lower and upper percentiles can also be different, such as −1 to 1, 0.5 to 1, 1 to 10, 1 to 99, and so on.

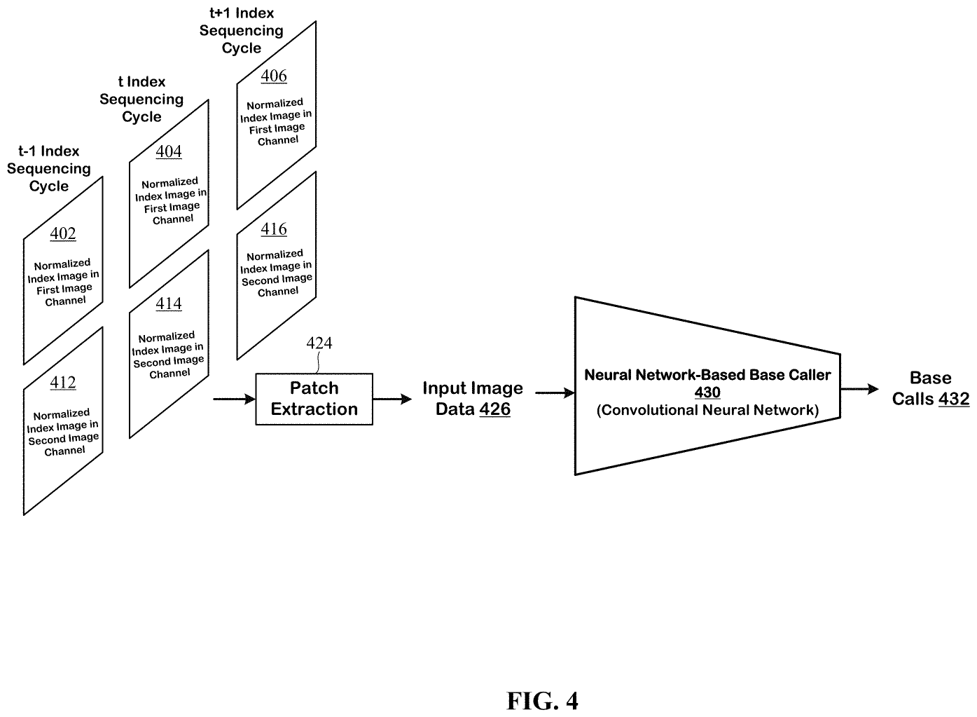

FIG. 4 depicts one implementation of processing normalized index images through the neural network-based base caller 430 for base calling.

In one implementation, the normalized index images 404, 414 from the current (time t) index sequencing cycle are accompanied with the normalized index images 402, 412 from the preceding (time t−1) index sequencing cycle and the normalized index images 406, 416 from the succeeding (time t+1) index sequencing cycle. These index images are normalized based on the intensity values of the index images in their corresponding flanking index sequencing cycles and their own respective intensity values, as discussed above.

The neural network-based base caller 430 processes the normalized index images 402, 412, 404, 414, 406, 416 through its convolution layers and produces an alternative representation, according to one implementation. The alternative representation is then used by an output layer (e.g., a softmax layer) for generating a base call for either just the current (time t) index sequencing cycle or each of the index sequencing cycles, i.e., the current (time t) index sequencing cycle, the preceding (time t−1) index sequencing cycle, and the succeeding (time t+1) index sequencing cycle. The resulting base calls form the index reads.

In one implementation, a patch extraction process 424 extracts patches from the normalized index images 402, 412, 404, 414, 406, 416 and generates input image data 426, as discussed above. Then, the extracted images patches in the input image data 426 are provided to the neural network-based base caller 430 as input.

In one implementation, the index images are normalized during training of the neural network-based base caller 430 as well as during inference.

Additional details about how the neural network-based base caller 430 performs base calling and the patch extraction process 424 can be found in US Provisional Patent Application No. 62/821,766, titled “ARTIFICIAL INTELLIGENCE-BASED SEQUENCING,” (Attorney Docket No. ILLM 1008-9/IP-1752-PRV), filed on Mar. 21, 2019, which is incorporated herein by reference.

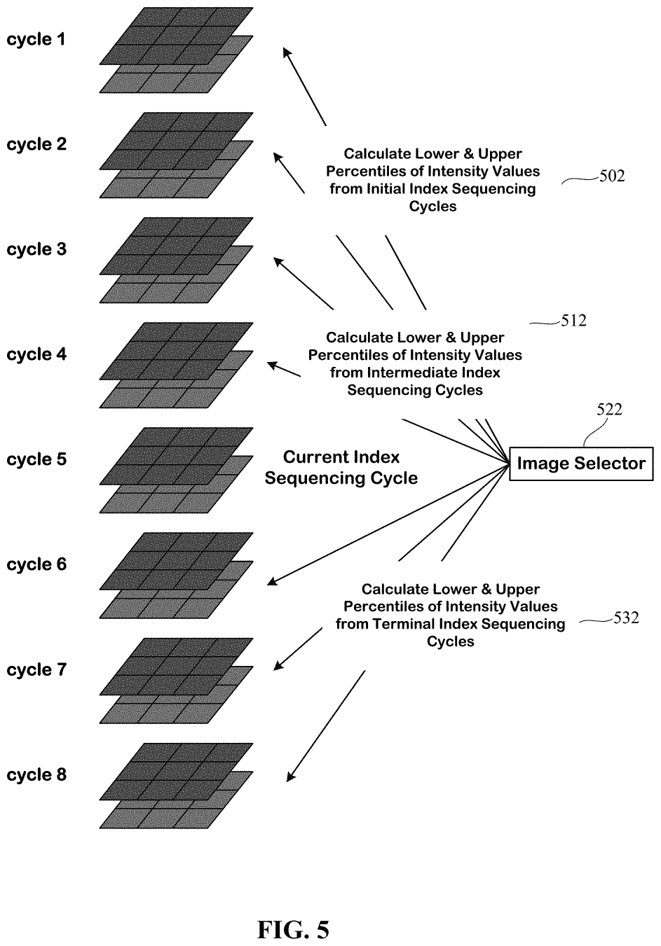

FIG. 5 shows one implementation of expanding the normalization of index images to non-current index sequencing cycles.

In other implementations, the index image from the current index sequencing cycle can be normalized based on (i) intensity values of index images from one or more non-current index sequencing cycles, and (ii) intensity values of index images from the current index sequencing cycle. The index images from the non-current index sequencing cycles can be selected by an image selector 522 and provided to the percentiles calculator 302 and the image normalizer 354 for normalization.

That is, the normalization 344 can expand beyond just flanking index sequencing cycles and does not always have to use immediately preceding or succeeding index sequencing cycles. For example, the non-current index sequencing cycles can comprise initial index sequencing cycles 502 (e.g., the first 2, 3, 5, 10, 20 index sequencing cycles). The non-current index sequencing cycles can comprise intermediate index sequencing cycles 512 (e.g., the middle 2, 3, 5, 10, 20 index sequencing cycles). The non-current index sequencing cycles can comprise terminal index sequencing cycles 532 (e.g., the last 2, 3, 5, 10, 20 index sequencing cycles).

Furthermore, the non-current index sequencing cycles can comprise a combination of the initial index sequencing cycles, the intermediate index sequencing cycles, and the terminal index sequencing cycles (e.g., the first and the fifth index sequencing cycles, the fifteenth and the twenty-third index sequencing cycles, and the eighteenth and the one-hundred forty-ninth index sequencing cycles).

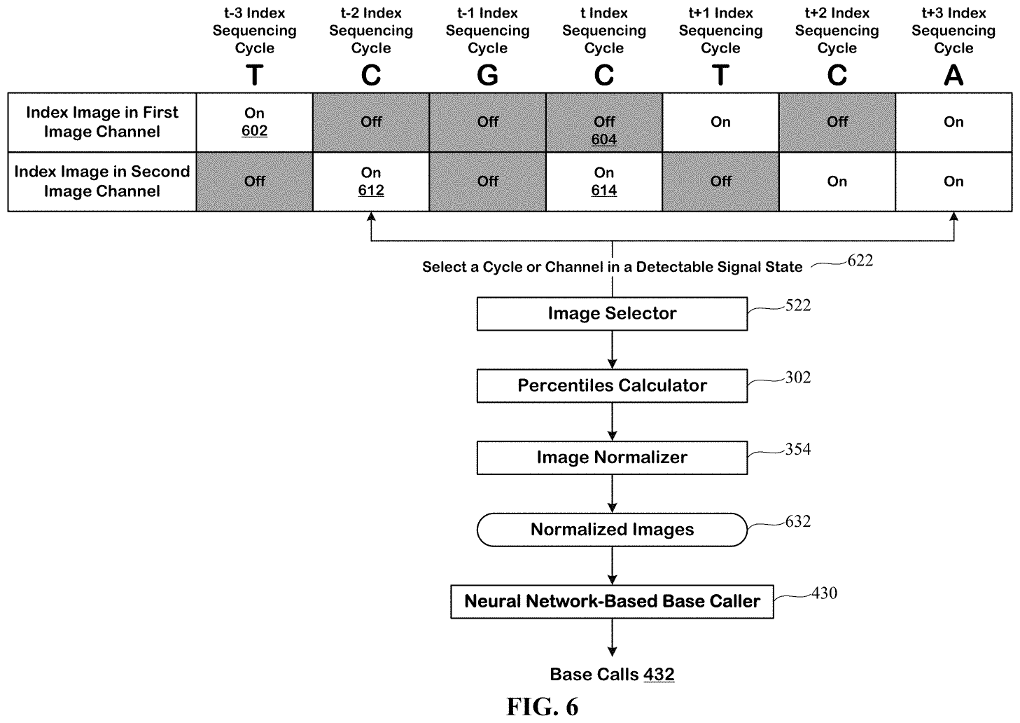

FIG. 6 illustrates one implementation of normalizing index images using at least one index image that depicts one or more nucleotides in the detectable signal state (i.e., on/detectable).

Regarding the detectable signal state, one avenue of differentiating between the different strategies for detecting nucleotide incorporation in a sequencing reaction using one fluorescent dye (or two or more dyes of same or similar excitation/emission spectra) is by characterizing the incorporations in terms of the presence or relative absence, or levels in between, of fluorescence transition that occurs during a sequencing cycle. As such, sequencing strategies can be exemplified by their fluorescent profile for a sequencing cycle. For strategies disclosed herein, “1” or “on” and “0” or “off” denotes a fluorescent state in which a nucleotide is in a “detectable signal state” (e.g., detectable by fluorescence) (1/on) or whether a nucleotide is in a dark state (e.g., not detected or minimally detected at an imaging step) (0/off). A “0” or “off” state does not necessarily refer to a total lack, or absence of signal. Although in some implementations there may be a total lack or absence of signal (e.g., fluorescence). Minimal or diminished fluorescence signal (e.g., background signal) is also contemplated to be included in the scope of a “0” or “off” state as long as a change in fluorescence from the first to the second image (or vice versa) can be reliably distinguished.

In the illustrated two-channel implementation of FIG. 6, nucleotide “G” is dark/off in both the index images, nucleotide “A” is on/detectable in both the index images, nucleotide “C” is dark/off in the first index image and on/detectable in the second index image, and nucleotide “T” is on/detectable in the first index image and dark/off in the second index image.

In one implementation, the image selector 522 selects 622 an index image from a non-current index sequencing cycle that is in the detectable signal state, and passes it to the percentiles calculator 302 and the image normalizer 354 to generate normalized images 632. The on/detectable index image can come from a non-current index sequencing cycle in which all the index images are in the detectable signal state (e.g., t+3 index sequencing cycle), or from a non-current index sequencing cycle in which only some of the index images are in the detectable signal state (e.g., t−2 index sequencing cycle).

In some implementations, many index images in the detectable signal state can be used for normalizing an index image.

In preferred implementations, on/detectable index images are selected across channels such that an index image in a first image channel (e.g., red channel) is normalized using one or more on/detectable index images in the first image channel and also one or more on/detectable index images in other image channels (e.g., green channel).

In other implementations, on/detectable index images are selected on a channel-by-channel basis such that an index image in a particular image channel is normalized using one or more on/detectable index images only in that particular image channel and not in different image channels. For example, the index image 604 in the first image channel can be normalized using the on/detectable index image 602 also in the first image channel (t−3 index sequencing cycle). Similarly, the index image 614 in the second image channel can be normalized using the on/detectable index image 612 also in the second image channel (t−2 index sequencing cycle).

### Normalization of Target Images

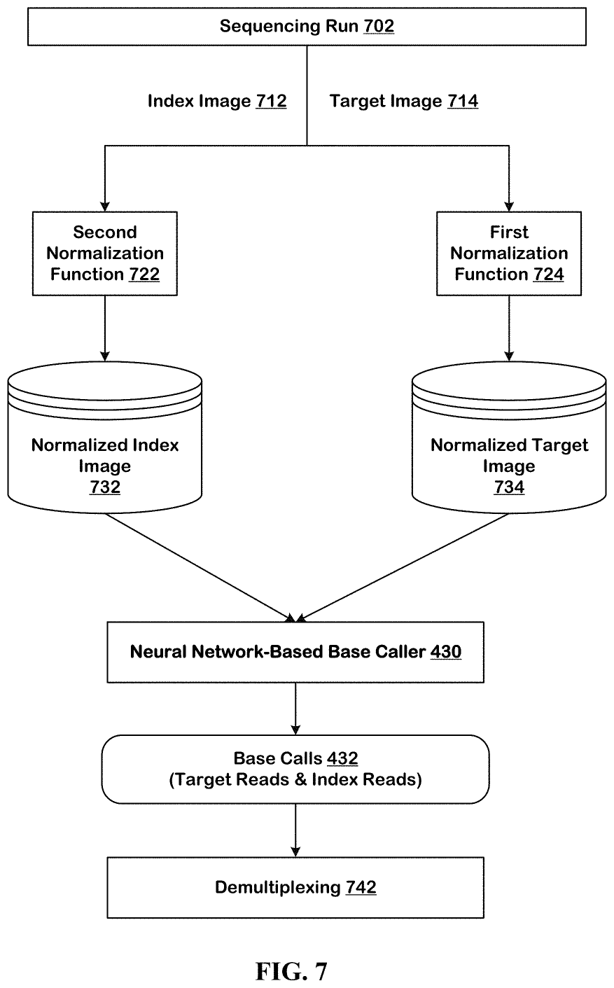

FIG. 7 depicts one implementation of base calling target sequences and index sequences. The target sequences are derived from a plurality of samples and coupled to the index sequences to form target-index sequences. Each index sequence is uniquely associated with a respective sample in the plurality of samples. The target-index sequences are pooled for sequencing during a sequencing run 702. The target sequences are sequenced during target sequencing cycles of the sequencing run and the index sequences are sequenced during index sequencing cycles of the sequencing run.

The technology disclosed normalizes the target images differently than it normalizes the index images. The target images depict intensity emissions generated as a result of nucleotide incorporation in the target sequences. The index images depict intensity emissions generated as a result of nucleotide incorporation in the index sequences.

For preprocessing a target image 714, the technology disclosed uses a first normalization function 724 that produces a normalized version 734 of the target image 714 from a current target sequencing cycle based only on intensity values of the target image 714. The first normalization function 724 calculates a lower percentile of the intensity values of the target image 714, and an upper percentile of the intensity values of the target image 714. In the normalized version 734 of the target image 714, a first percentage of normalized intensity values are below the lower percentile, a second percentage of the normalized intensity values are above the upper percentile, and a third percentage of the normalized intensity values are between the lower and upper percentiles.

For preprocessing an index image 712, the technology disclosed uses a second normalization function 722 that produces a normalized version 732 of the index image 712 from a current index sequencing cycle based on (i) intensity values of index images from one or more preceding index sequencing cycles, (ii) intensity values of index images from one or more succeeding index sequencing cycles, and (iii) intensity values of index images from the current index sequencing cycle.

The second normalization function 722 calculates a lower percentile of (i) the intensity values of the index images from the one or more preceding index sequencing cycles, (ii) the intensity values of the index images from the one or more succeeding index sequencing cycles, and (iii) the intensity values of the index images from the current index sequencing cycle, and an upper percentile of (i) the intensity values of the index images from the one or more preceding index sequencing cycles, (ii) the intensity values of the index images from the one or more succeeding index sequencing cycles, and (iii) the intensity values of the index images from the current index sequencing cycle. In the normalized version 732 of the index image 712, a first percentage of normalized intensity values are below the lower percentile, a second percentage of the normalized intensity values are above the upper percentile, and a third percentage of the normalized intensity values are between the lower and upper percentiles.

The technology disclosed processes normalized versions of the target images through the neural network-based base caller 430 and generates a base call for each of the target sequencing cycles, thereby producing target reads for the target sequences.

The technology disclosed processes normalized versions of the index images through the neural network-based base caller 430 and generates a base call for each of the index sequencing cycles, thereby producing index reads for the index sequences.

The technology disclosed performs demultiplexing 742 by classifying each target read of a target sequence as belonging to a particular sample in the plurality of samples based on a corresponding index read of an index sequence that is coupled to the target sequence.

### Augmentation

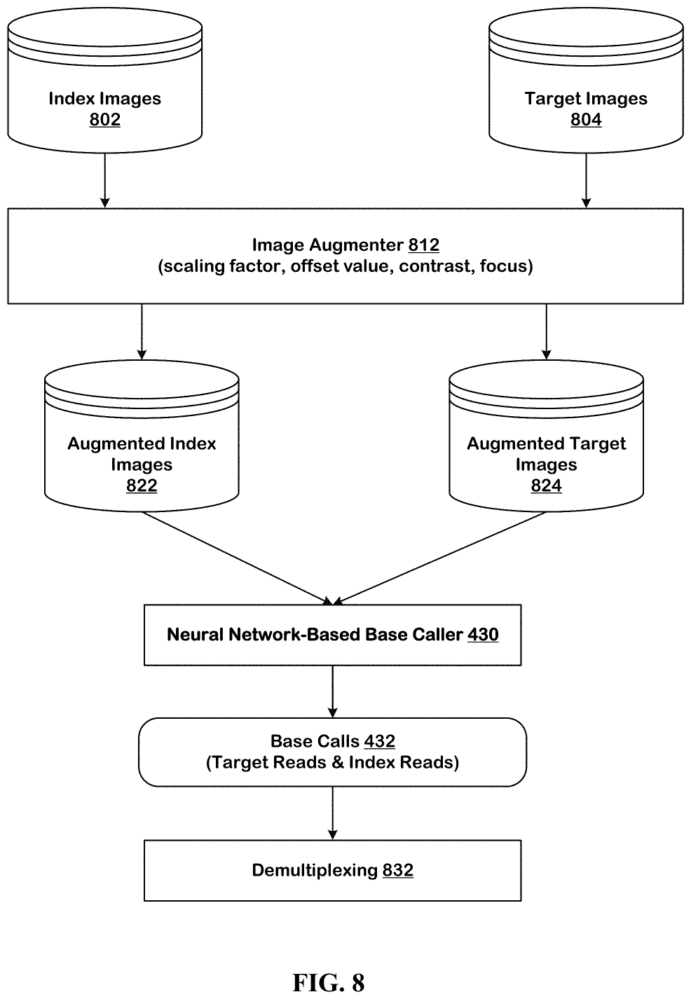

FIG. 8 illustrates one implementation of preprocessing that uses augmentation. An image augmenter 812 preprocesses the index images 802 and the target images 804 using an augmentation function. In one implementation, the image augmenter 812 multiplies the intensity values of the index images 802 and the target images 804 with a scaling factor and adds an offset value to the multiplication's result. In another implementation, the image augmenter 812 changes the contrast of the index images 802 and the target images 804. In yet another implementation, the image augmenter 812 changes the focus of the index images 802 and the target images 804.

The image augmenter 812 is configured with image augmentation logic to multiply intensity values of images with scaling factors and to add offset values to the results of the multiplication operations. The image augmenter 812 can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media).

In one implementation, the augmentation of the index images 802 and the target images 804 is performed only during the training of the neural network-based base caller and not during the inference.

The augmented index images 822 and the augmented target images 824 are processed through the neural network-based base caller 830 to generate a base call for each of the index sequencing cycles, thereby producing index reads for the index sequences, and to generate a base call for each of the target sequencing cycles, thereby producing target reads for the target sequences.

The technology disclosed performs demultiplexing 832 by classifying each target read of a target sequence as belonging to a particular sample in the plurality of samples based on a corresponding index read of an index sequence that is coupled to the target sequence.

### Example Preprocessing Results

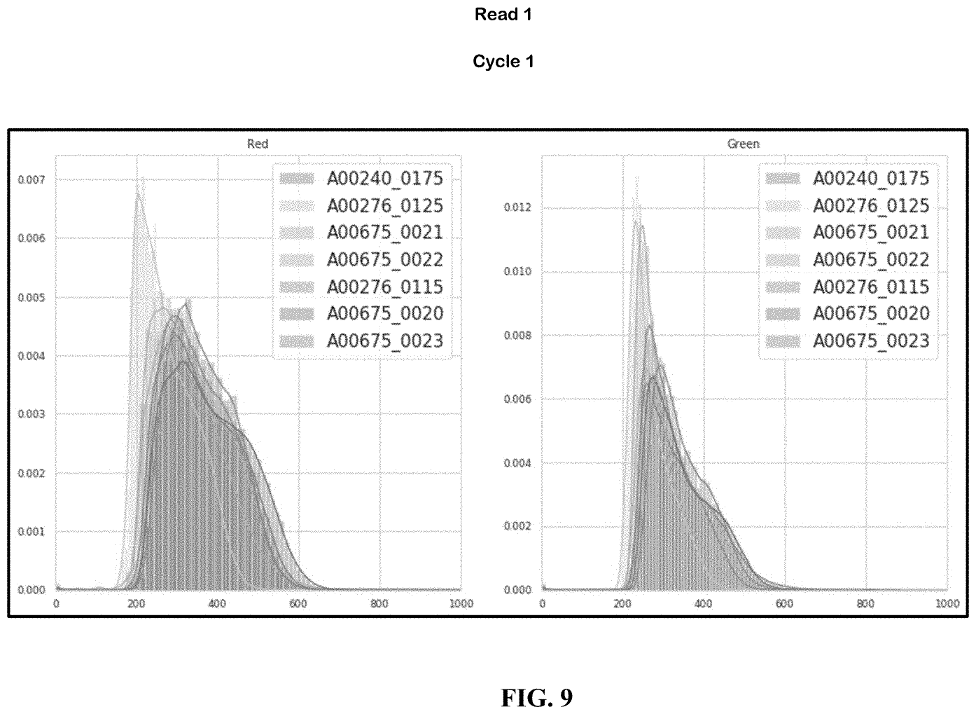

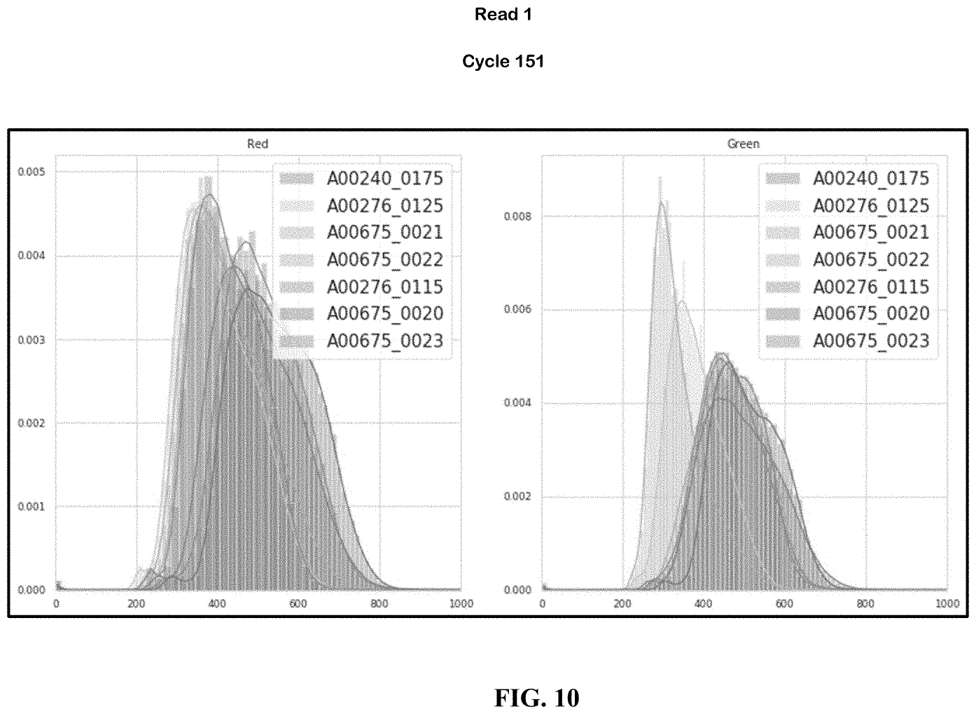

FIGS. 9 and 10 depict pixel intensity histograms of red and green images of two target sequencing cycles (cycles 1 and 151) of a first target read (Read 1).

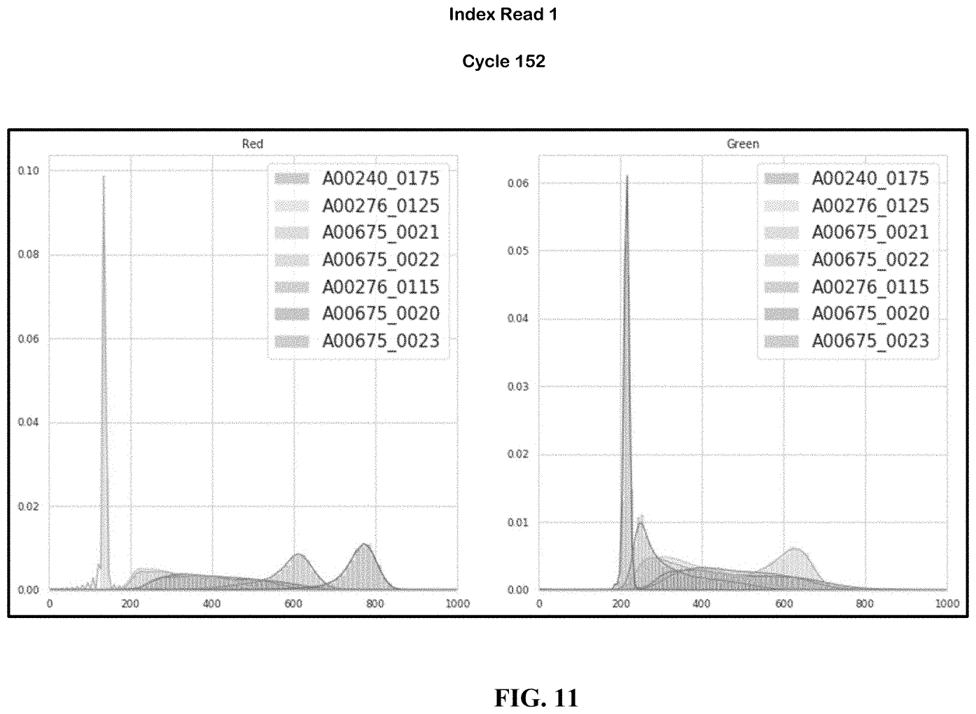

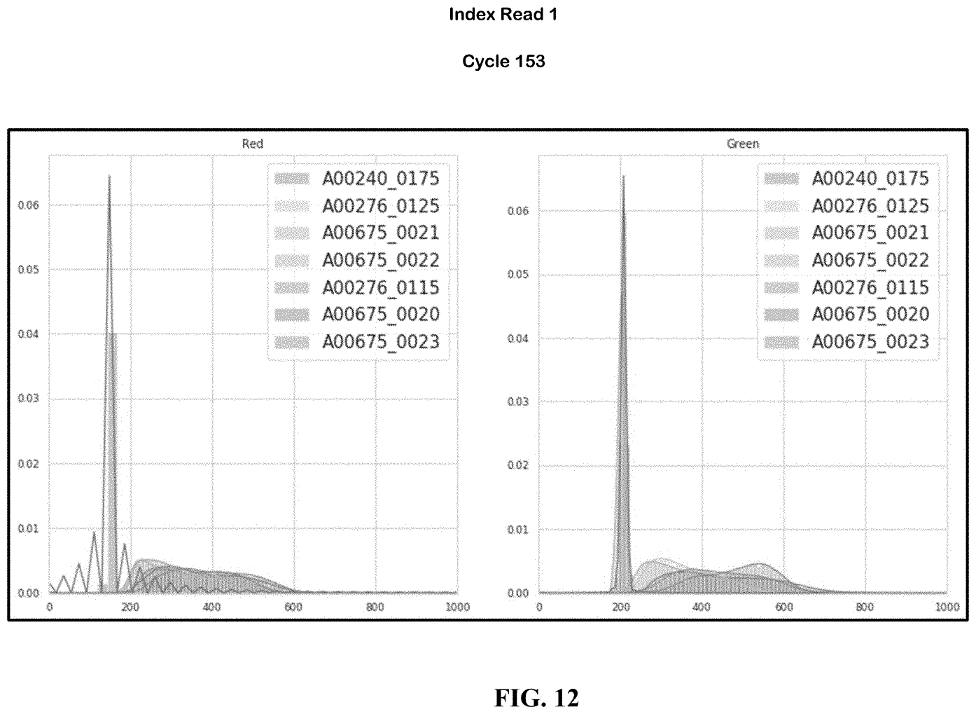

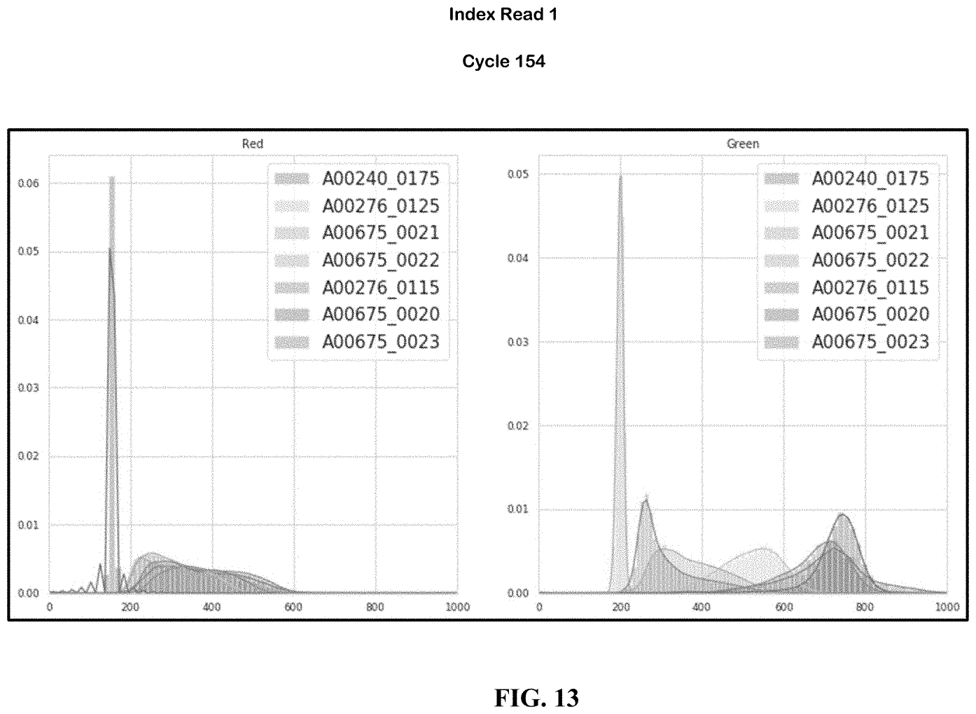

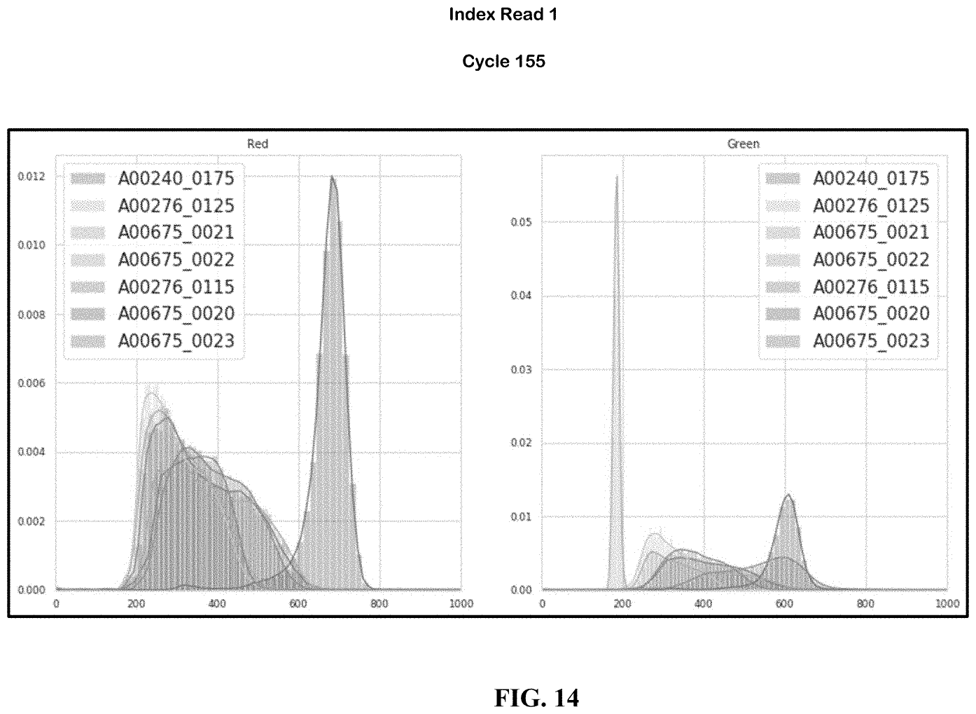

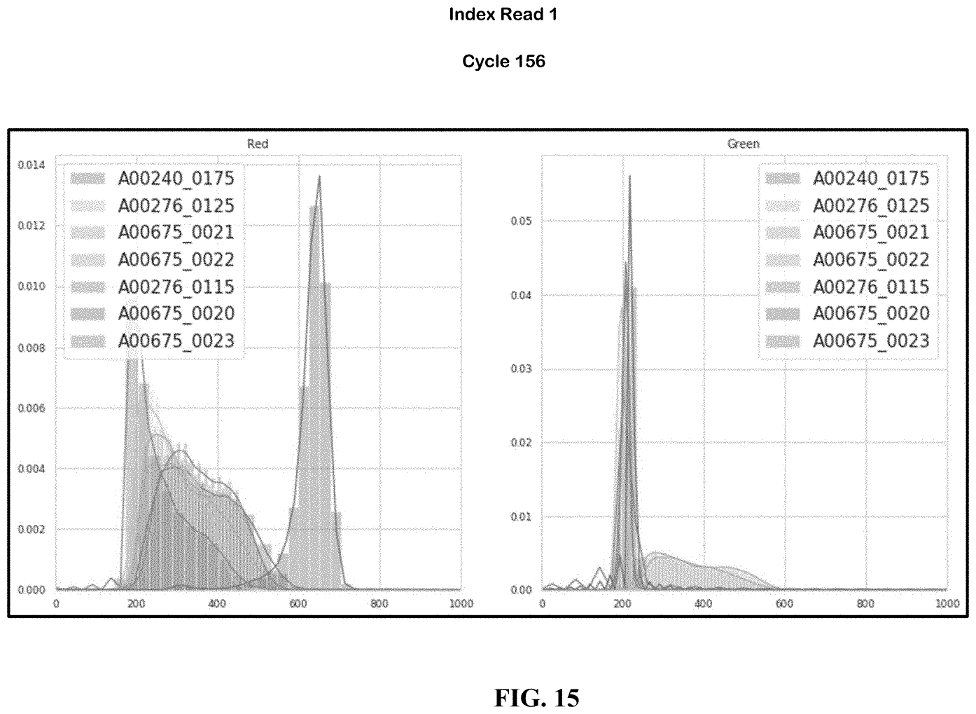

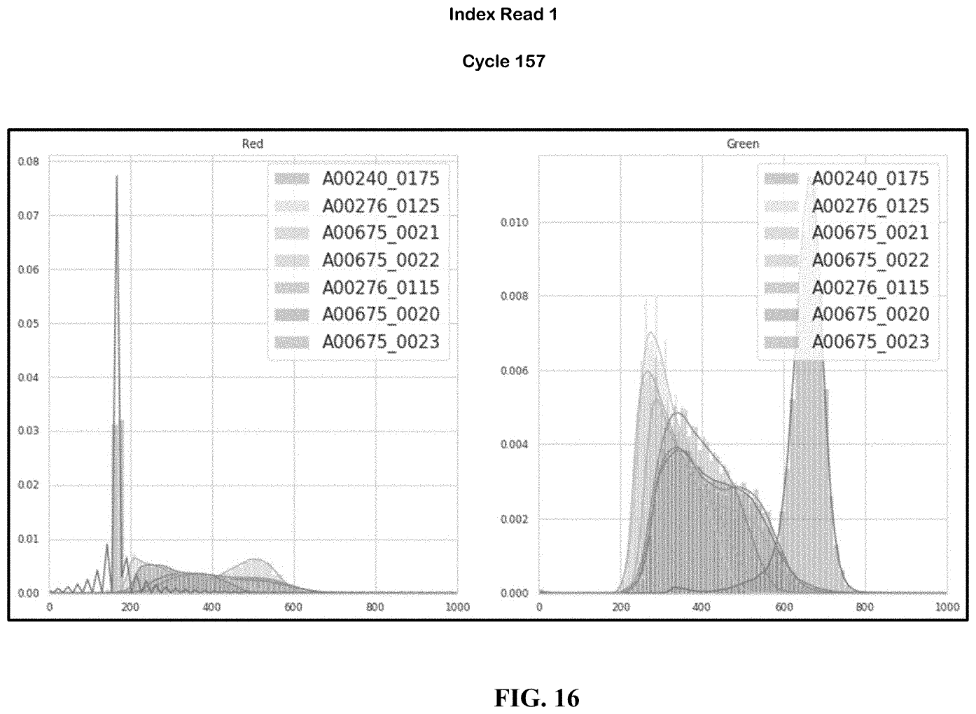

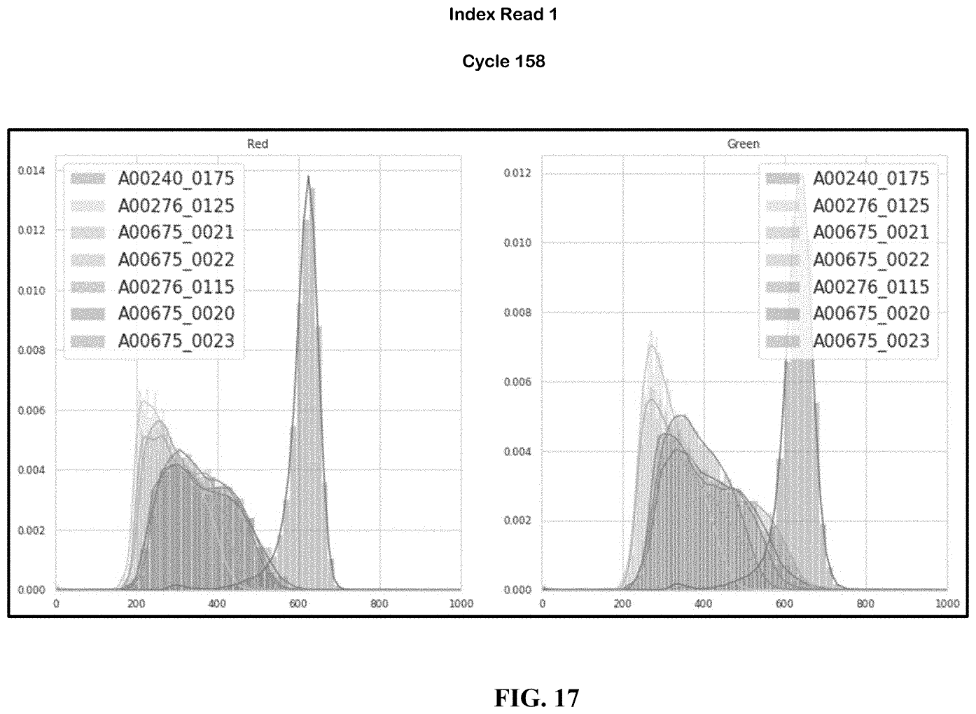

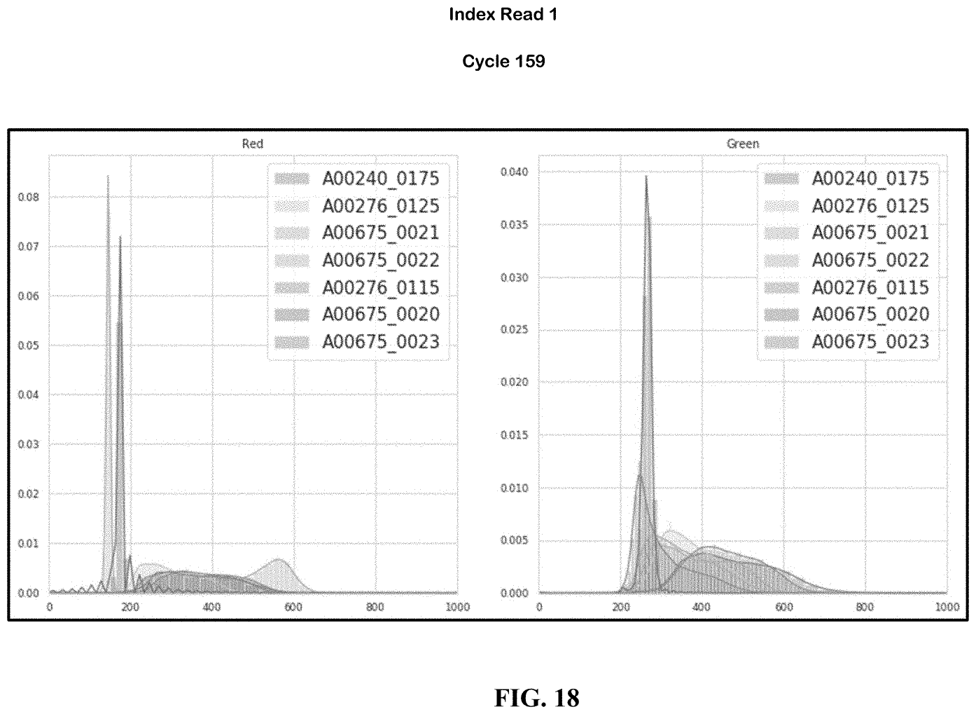

FIGS. 11, 12, 13, 14, 15, 16, 17, and 18 depict pixel intensity histograms of red and green images of eight index sequencing cycles (cycles 152, 153, 154, 155, 156, 157, 158, and 159) of a first index read (Index Read 1).

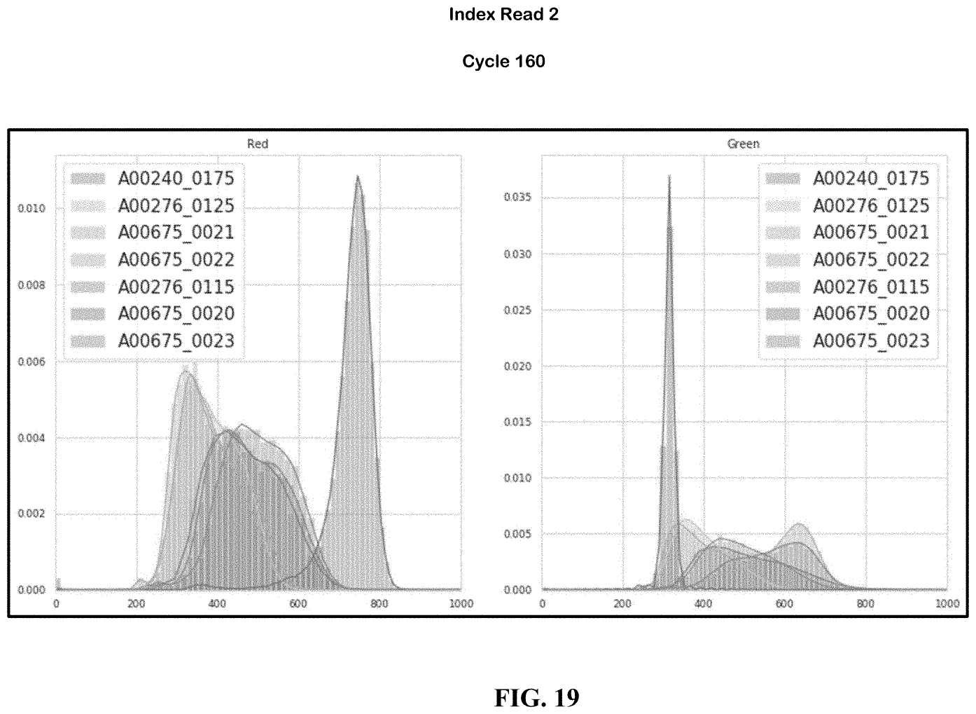

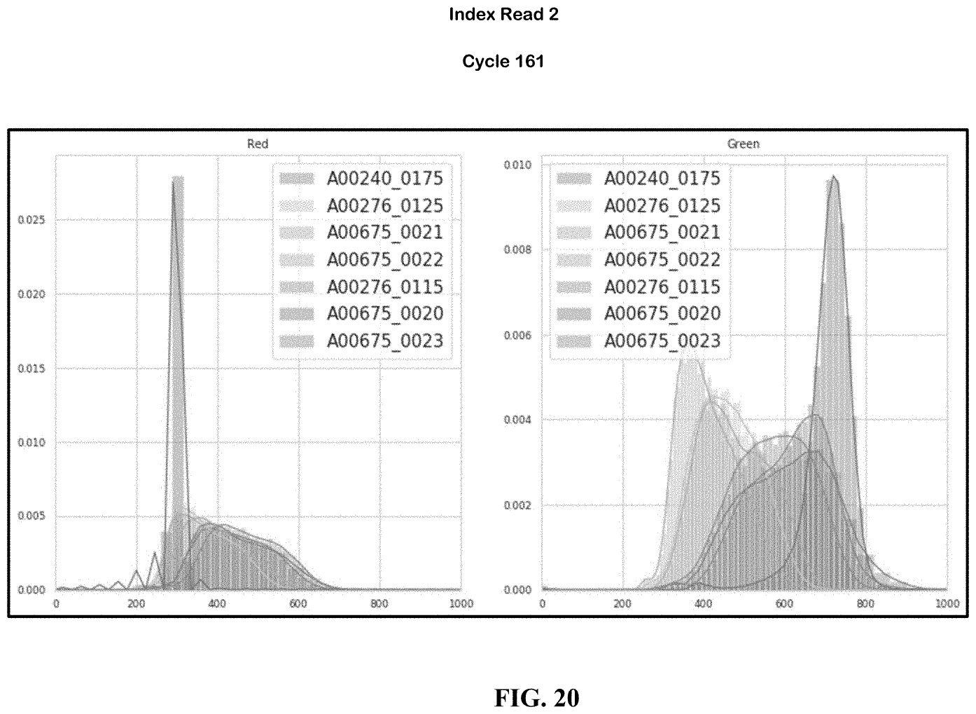

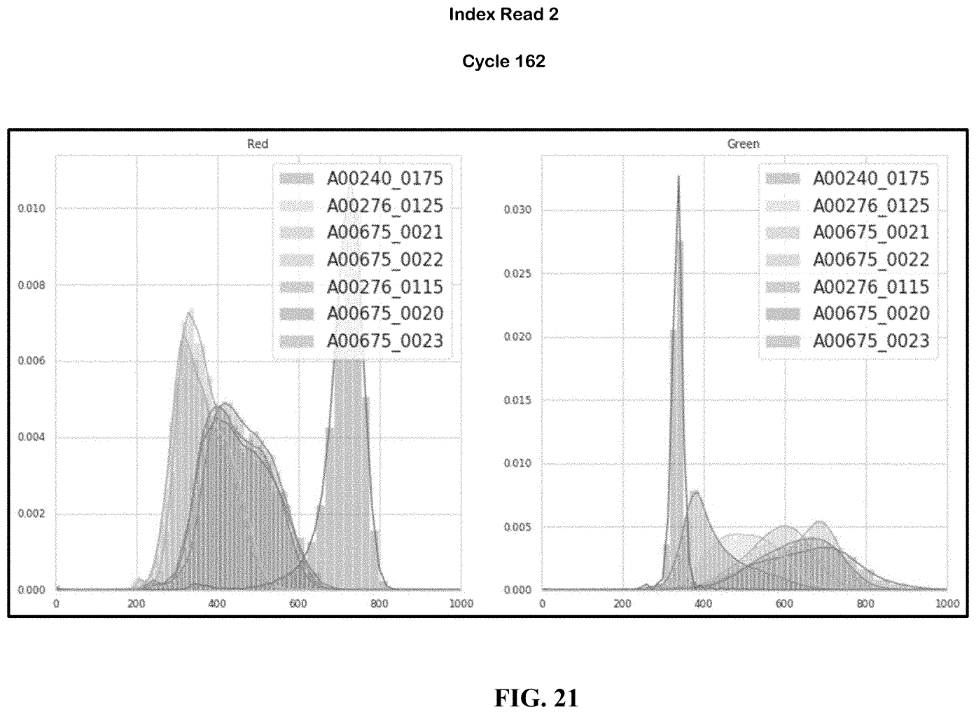

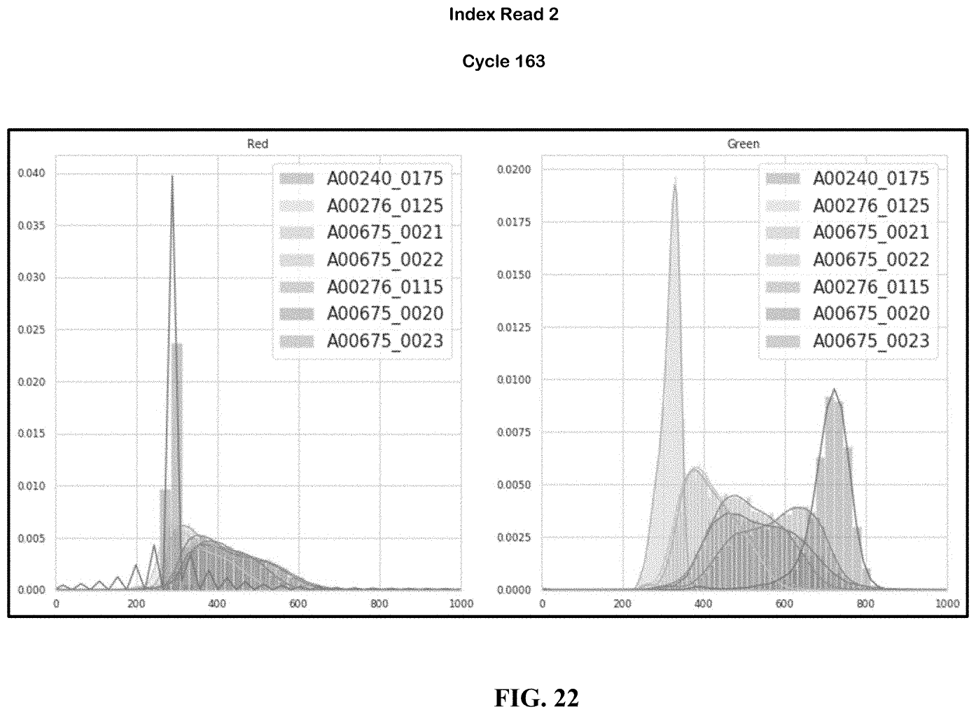

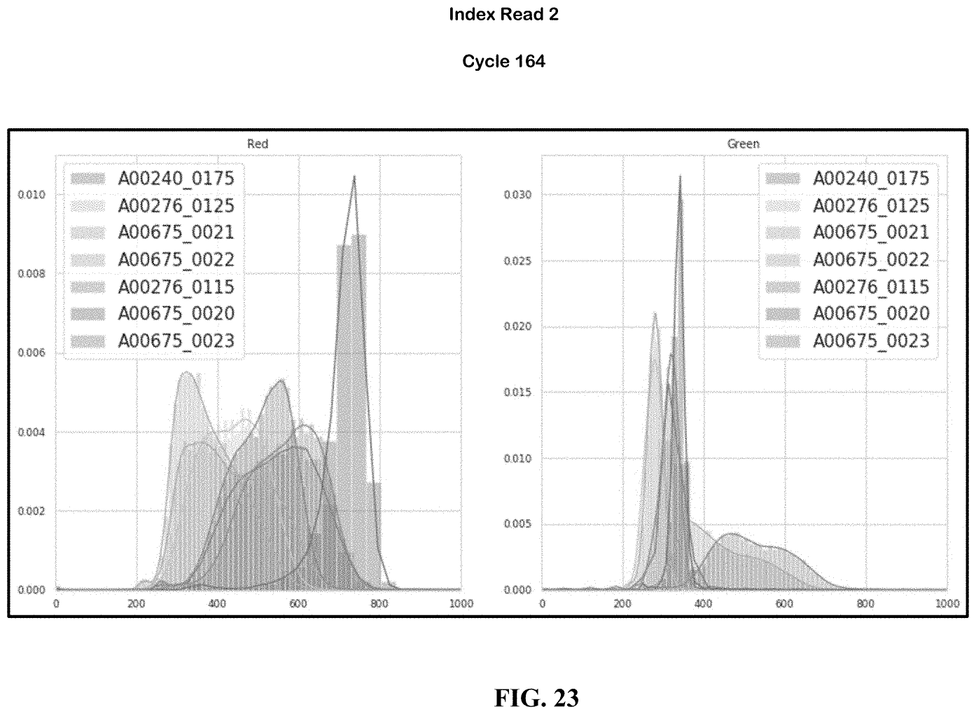

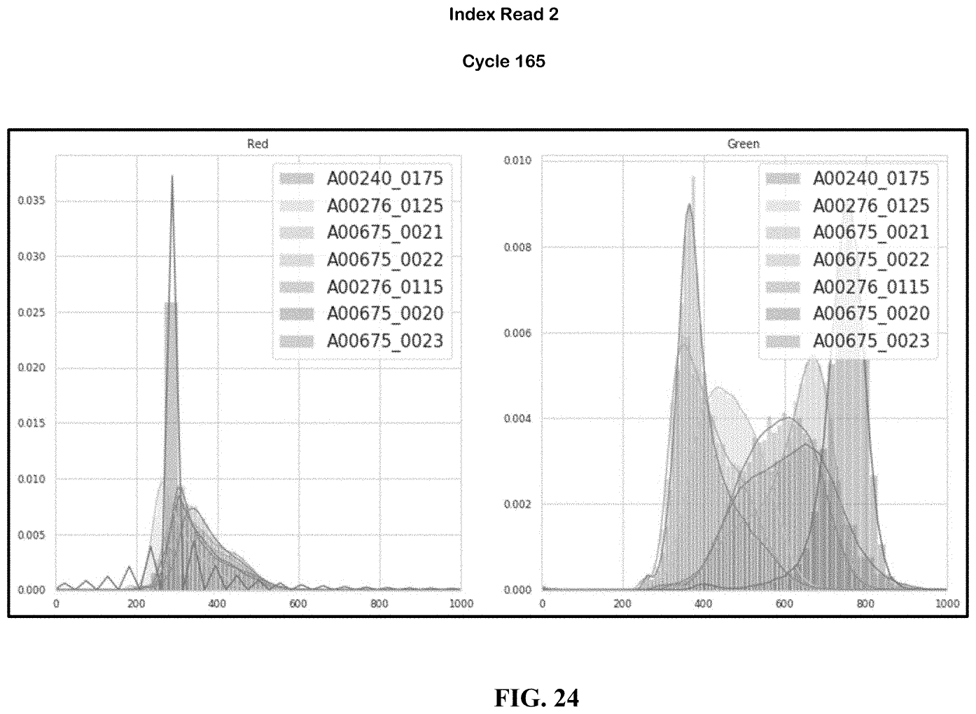

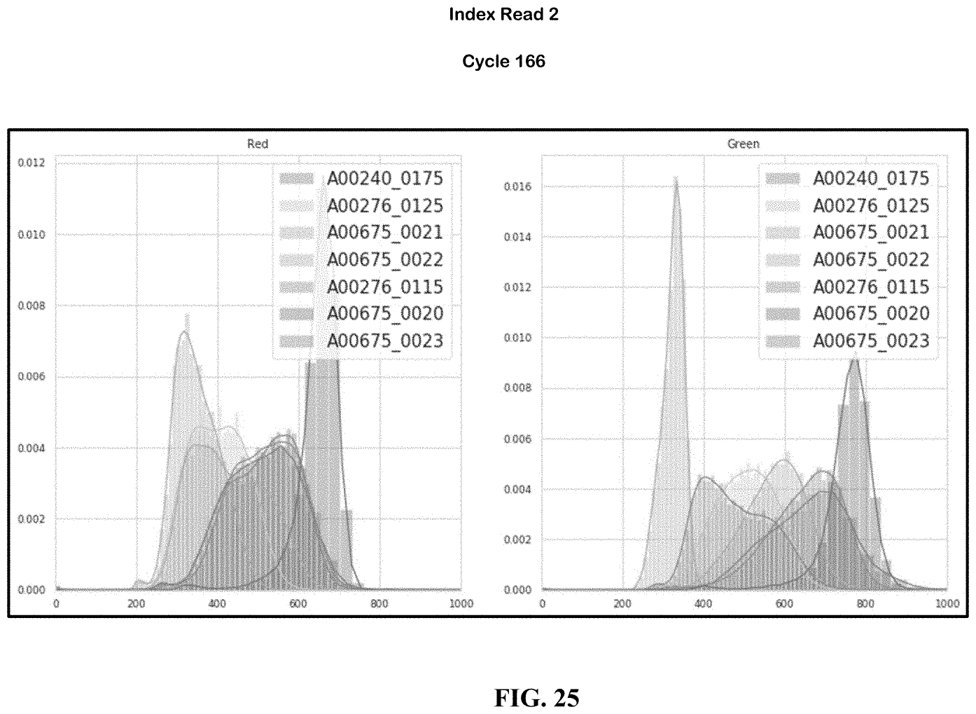

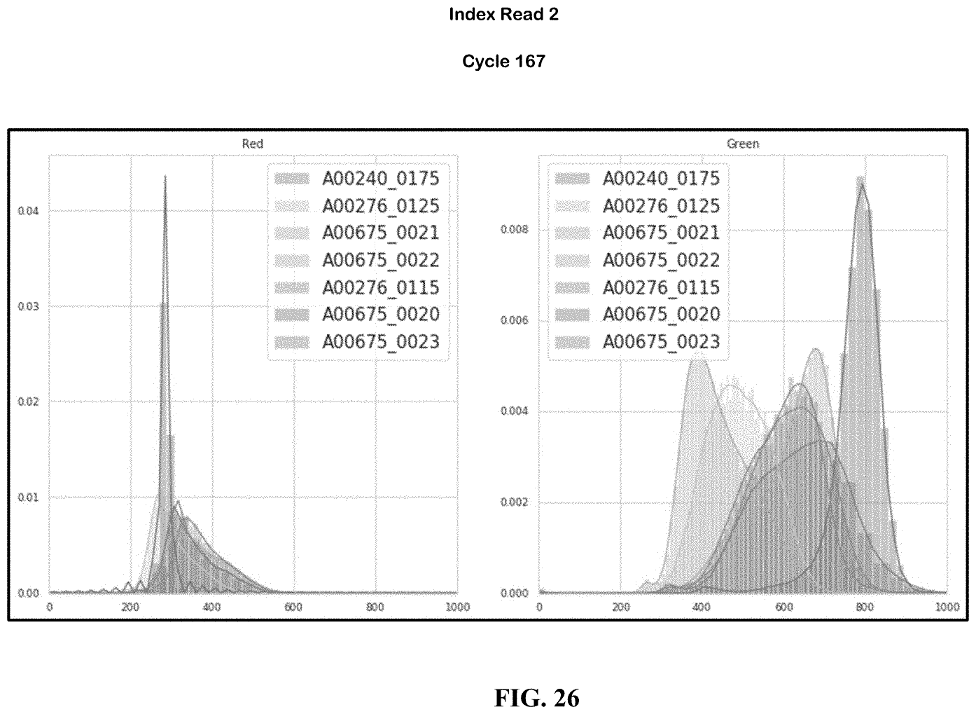

FIGS. 19, 20, 21, 22, 23, 24, 25, and 26 depict pixel intensity histograms of red and green images of eight index sequencing cycles (cycles 160, 161, 162, 163, 164, 165, 166, and 167) of a second index read (Index Read 2).

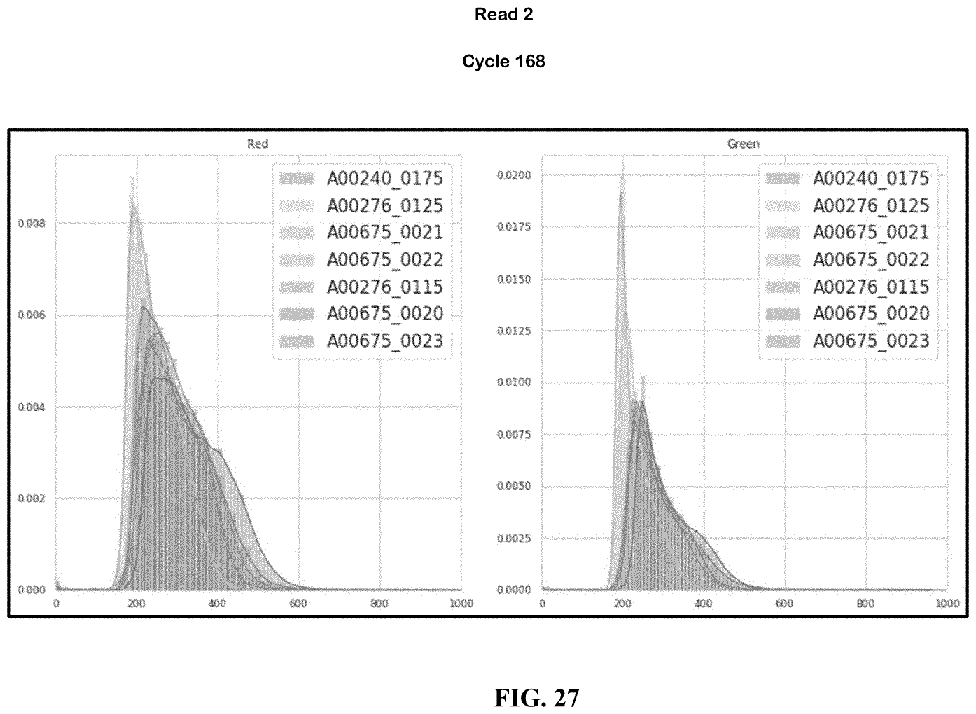

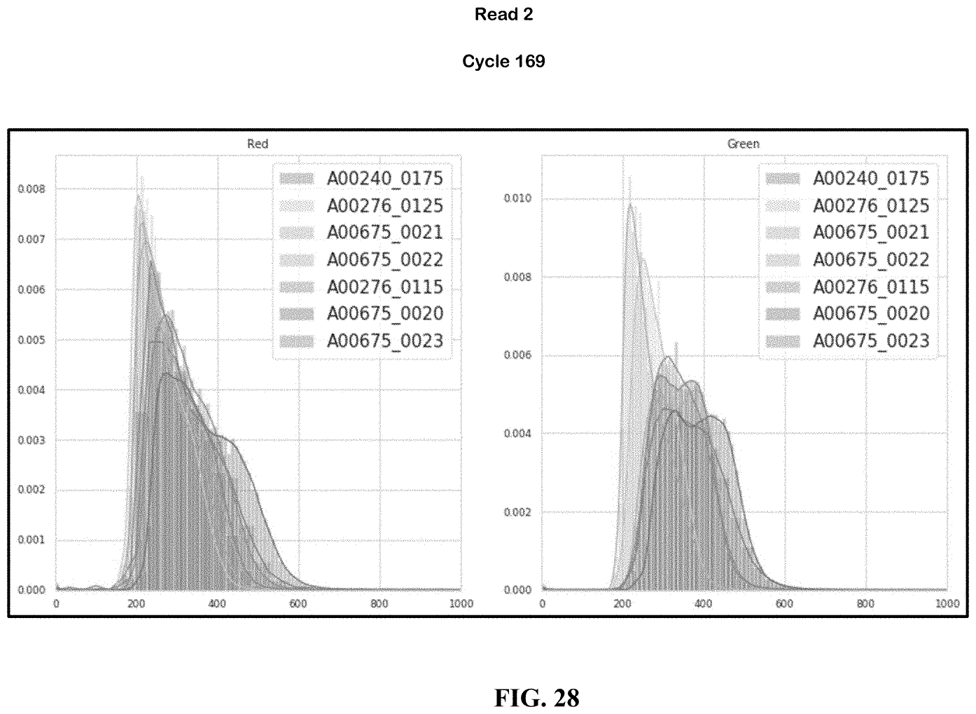

FIGS. 27 and 28 depict pixel intensity histograms of red and green images of two target sequencing cycles (cycles 168 and 169) of a second target read (Read 2).

So, Read 1 is followed by Index Read 1, which is followed by Index Read 2, and which is in turn followed by Read 2.

Here, each figure has two pixel intensity histograms for a given target or index sequencing cycle, one for the red image (on the left) and another for the green image (on the right). The x-axis of the pixel intensity histograms denotes the pixel intensities. The y-axis of the pixel intensity histograms denotes the pixel count or the pixel density. So, for example, if an image has 10,000 pixels, then a corresponding pixel intensity histogram depicts how frequently certain pixel intensities are found in the image.

The legends refer to names of seven different sequencing runs (e.g., A00240_0175, A00276_0125, A00675_0021, and so on), along with their corresponding color codes. The color codes convey how the pixel intensity distributions vary across the different sequencing runs.

The progression of the pixel intensity histograms from FIGS. 9 to 28 shows that the pixel intensity distribution variation across the target and index sequencing cycles is not substantial. This means that the pixel intensity values can be mixed to calculate the normalization parameters with the confidence that they are not far off from the appropriate value.

### Technical Effect and Performance Results as Objective Indicia of Inventiveness

The following discussion shows that normalizing and augmenting the index images improves the base calling accuracy of the neural network-based base caller 430 for index sequences. In particular, the following performance results provide an objective indicia of inventiveness of the technology disclosed with the base calling error increasing when the neural network-based base caller 430 does not use the disclosed normalization and augmentation techniques versus when the neural network-based base caller 430 does use the disclosed normalization and augmentation techniques.

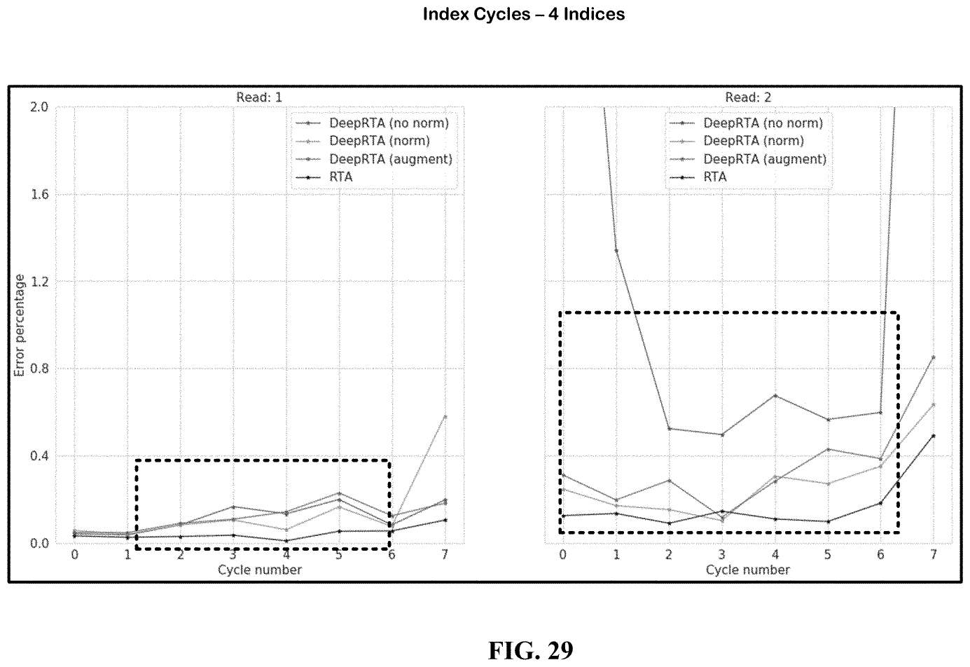

The graphs shown in FIGS. 29, 30, and 31 have four types of lines: a cyan line, a yellow line, a green line, and a black line.

The cyan line represents the index base calling performance of the neural network-based base caller 430 when the index images are NOT normalized (“DeepRTA (no norm)”).

The yellow line represents the index base calling performance of the neural network-based base caller 430 when the index images are normalized (“DeepRTA (norm)”).

The green line represents the index base calling performance of the neural network-based base caller 430 when the index images are augmented (“DeepRTA (augment)”).

The black line represents the index base calling performance of Illumina's non-neural network-based base caller called Real-Time Analysis (“RTA”). Additional details about RTA can be found in US Patent Publication No. 2012/0020537, titled “DATA PROCESSING SYSTEM AND METHODS,” (Attorney Docket No. ILLINC.174A), filed Jan. 13, 2011, which is incorporated herein by reference.

RTA is known to have good base calling accuracy for index sequences and therefore can be used a baseline for comparison.

Also, in the graphs, the x-axis represents the error percentage, which is an indication of the base calling accuracy, and the y-axis represents the cycle number of the index sequencing cycles. Furthermore, the graphs show two index reads, Read: 1 and Read: 2, each with seven index sequencing cycles.

FIG. 29 shows that for a sequencing run that uses four index sequences for multiplexing four samples, the index base calling performance of the neural network-based base caller 430 drops when the index images are not normalized (e.g., cyan line in index Read: 2).

The error percentage is relatively low when the index images are normalized (yellow line) and also when they are augmented (green line), as indicated by the dotted rectangles. Furthermore, the error percentage for the normalization and the augmentation implementations is along the lines of the error percentage of RTA.

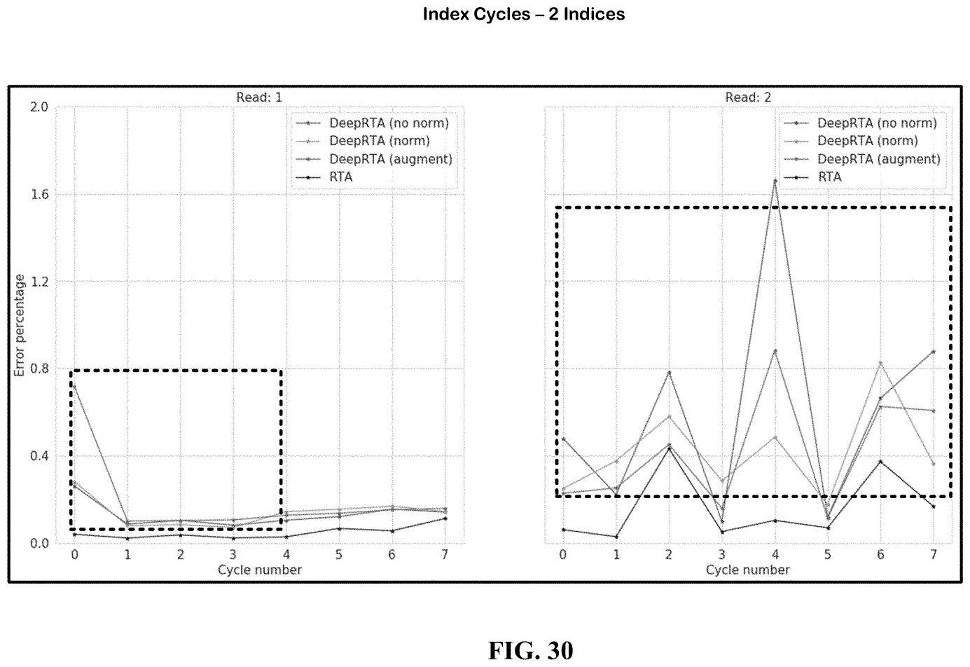

FIG. 30 shows that for a sequencing run that uses two index sequences for multiplexing two samples, the index base calling performance of the neural network-based base caller 430 drops when the index images are not normalized (e.g., cyan line in index Read: 2), as indicated by the dotted rectangles.

The error percentage is relatively low when the index images are normalized (yellow line) and also when they are augmented (green line). Furthermore, the error percentage for the normalization and the augmentation implementations is along the lines of the error percentage of RTA.

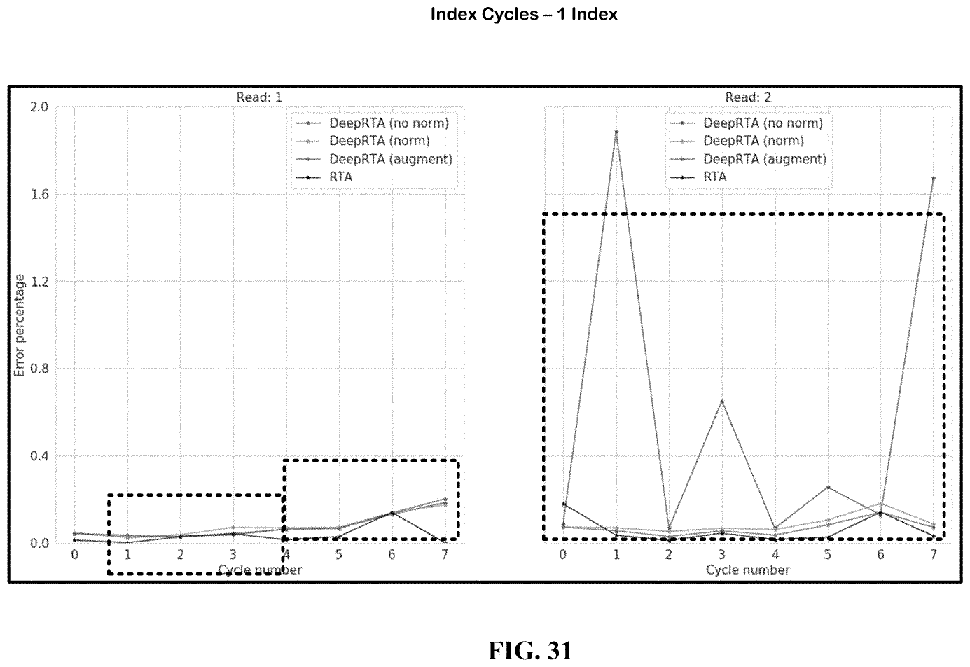

FIG. 31 shows that for a sequencing run that uses a single index sequence for sequencing a single sample, the index base calling performance of the neural network-based base caller 430 drops when the index images are not normalized (e.g., cyan line in index Read: 2) as indicated by the dotted rectangles.

The error percentage is relatively low when the index images are normalized (yellow line) and also when they are augmented (green line). Furthermore, the error percentage for the normalization and the augmentation implementations is along the lines of the error percentage of RTA.

### Base Calling Using Target Images and Index Images

FIG. 7 depicts one implementation of base calling target sequences and index sequences. The target sequences are derived from a plurality of samples and coupled to the index sequences to form target-index sequences. Each index sequence is uniquely associated with a respective sample in the plurality of samples. The target-index sequences are pooled for sequencing during a sequencing run 702. The target sequences are sequenced during target sequencing cycles of the sequencing run and the index sequences are sequenced during index sequencing cycles of the sequencing run.

In another implementation, the technology disclosed normalizes the target images and the index images in the same way. The target images depict intensity emissions generated as a result of nucleotide incorporation in the target sequences. The index images depict intensity emissions generated as a result of nucleotide incorporation in the index sequences.

For preprocessing an index image 712, the technology disclosed uses a second normalization function 722 that produces a normalized version 732 of the index image 712 from a current index sequencing cycle based on (i) intensity values of index images from one or more preceding index sequencing cycles, (ii) intensity values of index images from one or more succeeding index sequencing cycles, and (iii) intensity values of index images from the current index sequencing cycle.

The second normalization function 722 calculates a lower percentile of (i) the intensity values of the index images from the one or more preceding index sequencing cycles, (ii) the intensity values of the index images from the one or more succeeding index sequencing cycles, and (iii) the intensity values of the index images from the current index sequencing cycle, and an upper percentile of (i) the intensity values of the index images from the one or more preceding index sequencing cycles, (ii) the intensity values of the index images from the one or more succeeding index sequencing cycles, and (iii) the intensity values of the index images from the current index sequencing cycle. In the normalized version 732 of the index image 712, a first percentage of normalized intensity values are below the lower percentile, a second percentage of the normalized intensity values are above the upper percentile, and a third percentage of the normalized intensity values are between the lower and upper percentiles.

For preprocessing a target image 714, the technology disclosed also uses the second normalization function 722 that produces a normalized version 732 of the target image 714 from a current target sequencing cycle based on (i) intensity values of target images from one or more preceding target sequencing cycles, (ii) intensity values of target images from one or more succeeding target sequencing cycles, and (iii) intensity values of target images from the current target sequencing cycle.

The second normalization function 722 calculates a lower percentile of (i) the intensity values of the target images from the one or more preceding target sequencing cycles, (ii) the intensity values of the target images from the one or more succeeding target sequencing cycles, and (iii) the intensity values of the target images from the current target sequencing cycle, and an upper percentile of (i) the intensity values of the target images from the one or more preceding target sequencing cycles, (ii) the intensity values of the target images from the one or more succeeding target sequencing cycles, and (iii) the intensity values of the target images from the current target sequencing cycle. In the normalized version 732 of the target image 714, a first percentage of normalized intensity values are below the lower percentile, a second percentage of the normalized intensity values are above the upper percentile, and a third percentage of the normalized intensity values are between the lower and upper percentiles.

In one implementation, the normalization across target sequencing cycles also includes normalization across image channels within image data of the target sequencing cycles. For example, consider three target sequencing cycles: a first target sequencing cycle, a second target sequencing cycle, and a third target sequencing cycle. Also consider that each of the first, second, and third target sequencing cycles has two target images: a first target image (e.g., red target image) in a first image channel (e.g., red channel) and a second target image (e.g., green target image) in a second image channel (e.g., green channel). A red target image from the second target sequencing cycle is normalized based on (i) intensity values of red and green images from the first target sequencing cycle, (ii) intensity values of red and green images from the third target sequencing cycle, and (iii) intensity values of red and green images from the second target sequencing cycle. A green target image from the second target sequencing cycle is normalized based on (i) intensity values of red and green images from the first target sequencing cycle, (ii) intensity values of red and green images from the third target sequencing cycle, and (iii) intensity values of red and green images from the second target sequencing cycle.

The technology disclosed processes normalized versions of the target images through the neural network-based base caller 430 and generates a base call for each of the target sequencing cycles, thereby producing target reads for the target sequences.

The technology disclosed processes normalized versions of the index images through the neural network-based base caller 430 and generates a base call for each of the index sequencing cycles, thereby producing index reads for the index sequences.

In one implementation, preprocessing of the target images and the index images using the second normalization function 722 occurs during training of the neural network-based base caller as well as during inference.

The technology disclosed performs demultiplexing 742 by classifying each target read of a target sequence as belonging to a particular sample in the plurality of samples based on a corresponding index read of an index sequence that is coupled to the target sequence.

### Computer System

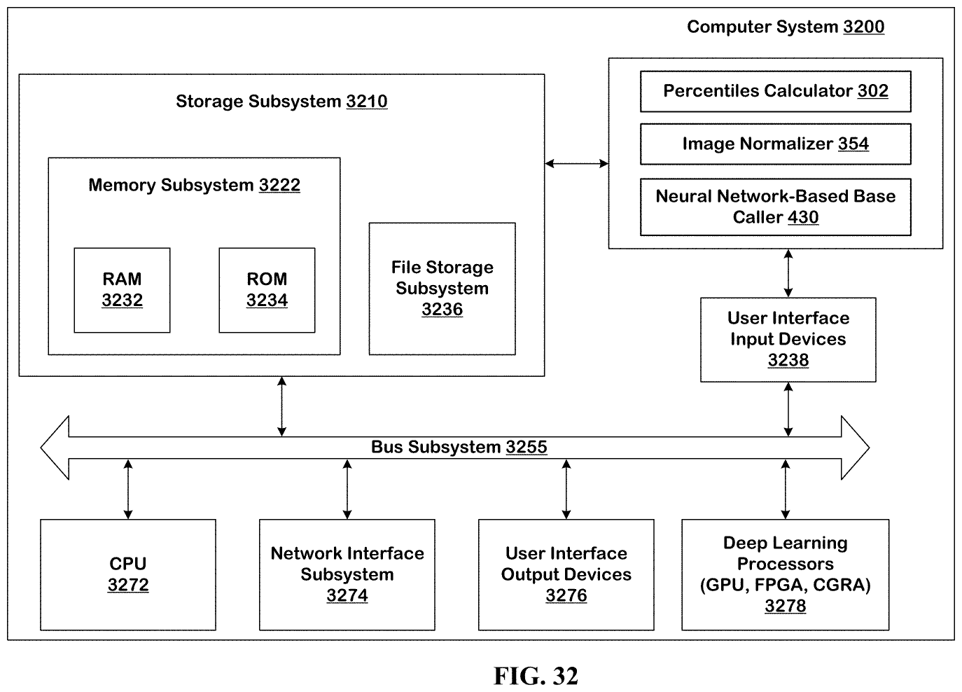

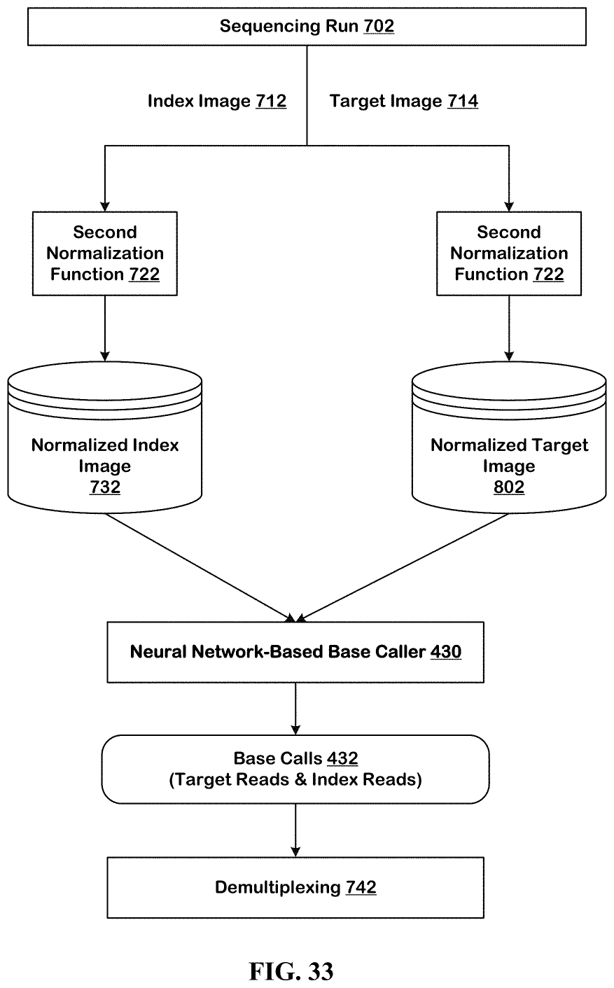

FIG. 32 is a computer system 3200 that can be used to implement the technology disclosed. Computer system 3200 includes at least one central processing unit (CPU) 3272 that communicates with a number of peripheral devices via bus subsystem 3255. These peripheral devices can include a storage subsystem 3210 including, for example, memory devices and a file storage subsystem 3236, user interface input devices 3238, user interface output devices 3276, and a network interface subsystem 3274. The input and output devices allow user interaction with computer system 3200. Network interface subsystem 3274 provides an interface to outside networks, including an interface to corresponding interface devices in other computer systems.

In one implementation, the percentiles calculator 302, the image normalizer 354, and the neural network-based base caller 430 are communicably linked to the storage subsystem 3210 and the user interface input devices 3238.

User interface input devices 3238 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system 3200.

User interface output devices 3276 can include a display subsystem, a printer, a fax machine, or non-visual displays such as audio output devices. The display subsystem can include an LED display, a cathode ray tube (CRT), a flat-panel device such as a liquid crystal display (LCD), a projection device, or some other mechanism for creating a visible image. The display subsystem can also provide a non-visual display such as audio output devices. In general, use of the term “output device” is intended to include all possible types of devices and ways to output information from computer system 3200 to the user or to another machine or computer system.

Storage subsystem 3210 stores programming and data constructs that provide the functionality of some or all of the modules and methods described herein. These software modules are generally executed by deep learning processors 3278.

Deep learning processors 3278 can be graphics processing units (GPUs), field-programmable gate arrays (FPGAs), application-specific integrated circuits (ASICs), and/or coarse-grained reconfigurable architectures (CGRAs). Deep learning processors 3278 can be hosted by a deep learning cloud platform such as Google Cloud Platform™, Xilinx™, and Cirrascale™. Examples of deep learning processors 3278 include Google's Tensor Processing Unit (TPU)™, rackmount solutions like GX4 Rackmount Series™, GX32 Rackmount Series™ NVIDIA DGX-1™, Microsoft′ Stratix V FPGA™, Graphcore's Intelligent Processor Unit (IPU)™, Qualcomm's Zeroth Platform™ with Snapdragon processors™, NVIDIA's Volta™ NVIDIA's DRIVE PX™, NVIDIA's JETSON TX1/TX2 MODULE™, Intel's Nirvana™ Movidius VPU™, Fujitsu DPI™, ARM's DynamiclQ™, IBM TrueNorth™, and others.

Memory subsystem 3222 used in the storage subsystem 3210 can include a number of memories including a main random access memory (RAM) 3232 for storage of instructions and data during program execution and a read only memory (ROM) 3234 in which fixed instructions are stored. A file storage subsystem 3236 can provide persistent storage for program and data files, and can include a hard disk drive, a floppy disk drive along with associated removable media, a CD-ROM drive, an optical drive, or removable media cartridges. The modules implementing the functionality of certain implementations can be stored by file storage subsystem 3236 in the storage subsystem 3210, or in other machines accessible by the processor.

Bus subsystem 3255 provides a mechanism for letting the various components and subsystems of computer system 3200 communicate with each other as intended. Although bus subsystem 3255 is shown schematically as a single bus, alternative implementations of the bus subsystem can use multiple busses.

Computer system 3200 itself can be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the description of computer system 3200 depicted in FIG. 32 is intended only as a specific example for purposes of illustrating the preferred implementations of the present invention. Many other configurations of computer system 3200 are possible having more or less components than the computer system depicted in FIG. 32.

### Particular Implementations

We describe various implementations of artificial intelligence-based base calling of index sequences. One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections—these recitations are hereby incorporated forward by reference into each of the following implementations.

In one implementation, we disclose an artificial intelligence-based method of base calling index sequences. The method includes accessing index images generated for the index sequences during index sequencing cycles of a sequencing run. The index images depict intensity emissions generated as a result of nucleotide incorporation in the index sequences during the sequencing run.

The method includes preprocessing the index images using a normalization function that produces a normalized version of an index image from a current index sequencing cycle based on (i) intensity values of index images from one or more preceding index sequencing cycles, (ii) intensity values of index images from one or more succeeding index sequencing cycles, and (iii) intensity values of index images from the current index sequencing cycle.

The method further includes processing normalized versions of the index images through a neural network-based base caller and generating a base call for each of the index sequencing cycles, thereby producing index reads for the index sequences.

The method described in this section and other sections of the technology disclosed can include one or more of the following features and/or features described in connection with additional methods disclosed. In the interest of conciseness, the combinations of features disclosed in this application are not individually enumerated and are not repeated with each base set of features. The reader will understand how features identified in these implementations can readily be combined with sets of base features identified in other implementations.

In one implementation, the normalization function calculates a lower percentile of (i) the intensity values of the index images from the one or more preceding index sequencing cycles, (ii) the intensity values of the index images from the one or more succeeding index sequencing cycles, and (iii) the intensity values of the index images from the current index sequencing cycle, and an upper percentile of (i) the intensity values of the index images from the one or more preceding index sequencing cycles, (ii) the intensity values of the index images from the one or more succeeding index sequencing cycles, and (iii) the intensity values of the index images from the current index sequencing cycle, such that, in the normalized version of the index image, a first percentage of normalized intensity values are below the lower percentile, a second percentage of the normalized intensity values are above the upper percentile, and a third percentage of the normalized intensity values are between the lower and upper percentiles.

In one implementation, taken together, nucleotides depicted by the index images from the current, preceding, and succeeding index sequencing cycles are cumulatively more diverse than nucleotides depicted only by the index images from the current index sequencing cycle. In some implementations, at least one index image in the index images from the preceding and succeeding index sequencing cycles depicts one or more nucleotides in a detectable signal state.

In one implementation, the nucleotides depicted by the index images from the current index sequencing cycle are low-complexity patterns in which some of four bases A, C, T, and G are represented at a frequency of less than 15%, 10%, or 5% of all the nucleotides.

In one implementation, taken together, the nucleotides depicted by the index images from the current, preceding, and succeeding index sequencing cycles cumulatively form high-complexity patterns in which each of the four bases A, C, T, and G is represented at a frequency of at least 20%, 25%, or 30% of all the nucleotides.

In one implementation, the method includes preprocessing the index images using the normalization function during training of the neural network-based base caller as well as during inference.

In one implementation, the method includes preprocessing the index images using an augmentation function that produces an augmented version of an index image by multiplying intensity values of the index image with a scaling factor and adding an offset value to the multiplication's result. The method further includes processing augmented versions of the index images through the neural network-based base caller and generating a base call for each of the index sequencing cycles, thereby producing index reads for the index sequences.

In one implementation, the method includes preprocessing the index images using the augmentation function only during the training of the neural network-based base caller and not during the inference.

In one implementation, the method includes preprocessing the index images using the normalization function that produces the normalized version of the index image from the current index sequencing cycle based on (i) intensity values of index images from one or more non-current index sequencing cycles, and (ii) intensity values of index images from the current index sequencing cycle. In some implementations, the non-current index sequencing cycles comprise initial index sequencing cycles of the sequencing. In other implementations, the non-current index sequencing cycles comprise intermediate index sequencing cycles of the sequencing. In some other implementations, the non-current index sequencing cycles comprise terminal index sequencing cycles of the sequencing. In yet other implementations, the non-current index sequencing cycles comprise a combination of the initial index sequencing cycles, the intermediate index sequencing cycles, and the terminal index sequencing cycles.

In one implementation, at least one index image from the non-current index sequencing cycles depicts one or more nucleotides in the detectable signal state.

Other implementations of the method described in this section can include a non-transitory computer readable storage medium storing instructions executable by a processor to perform any of the methods described above. Yet another implementation of the method described in this section can include a system including memory and one or more processors operable to execute instructions, stored in the memory, to perform any of the methods described above.

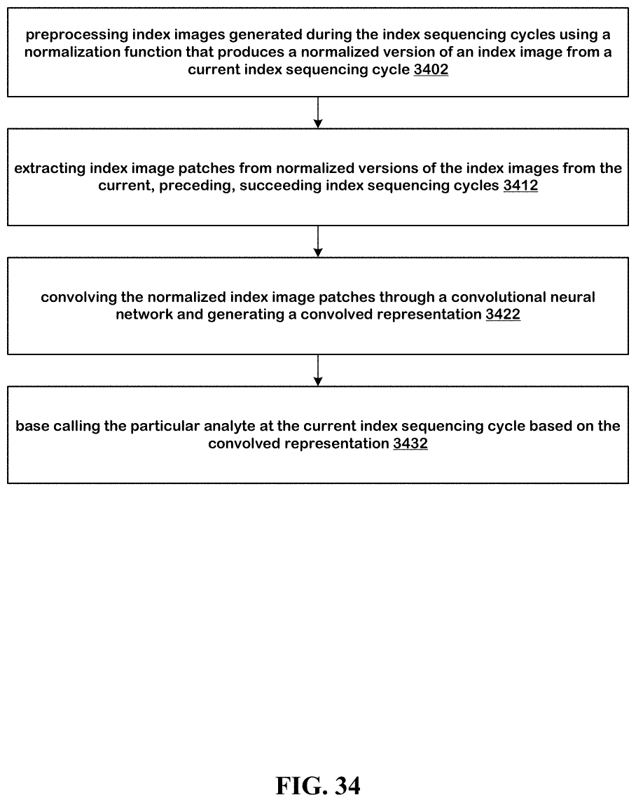

FIG. 34 is one implementation of a flow chart of an artificial intelligence-based method of base calling analytes at index sequencing cycles of a sequencing run. At action 3402, the method includes preprocessing index images generated during the index sequencing cycles using a normalization function that produces a normalized version of an index image from a current index sequencing cycle based on (i) intensity values of index images from one or more preceding index sequencing cycles, (ii) intensity values of index images from one or more succeeding index sequencing cycles, and (iii) intensity values of index images from the current index sequencing cycle.

For a particular analyte being base called at the current index sequencing cycle, at action 3412, the method includes extracting index image patches from normalized versions of the index images from the current, preceding, succeeding index sequencing cycles, such that, each normalized index image patch depicts intensity emissions of the particular analyte, of some adjacent analytes, and of their surrounding background generated as a result of nucleotide incorporation in corresponding index sequences of the particular analyte and the adjacent analytes during the current index sequencing cycle.

The method further includes, at action 3422, convolving the normalized index image patches through a convolutional neural network and generating a convolved representation.

The method further includes, at action 3432, base calling the particular analyte at the current index sequencing cycle based on the convolved representation.

Each of the features discussed in the particular implementation section for other implementations apply equally to this implementation. As indicated above, all the other features are not repeated here and should be considered repeated by reference. The reader will understand how features identified in these implementations can readily be combined with sets of base features identified in other implementations. Other implementations of the method described in this section can include a non-transitory computer readable storage medium storing instructions executable by a processor to perform any of the methods described above. Yet another implementation of the method described in this section can include a system including memory and one or more processors operable to execute instructions, stored in the memory, to perform any of the methods described above.

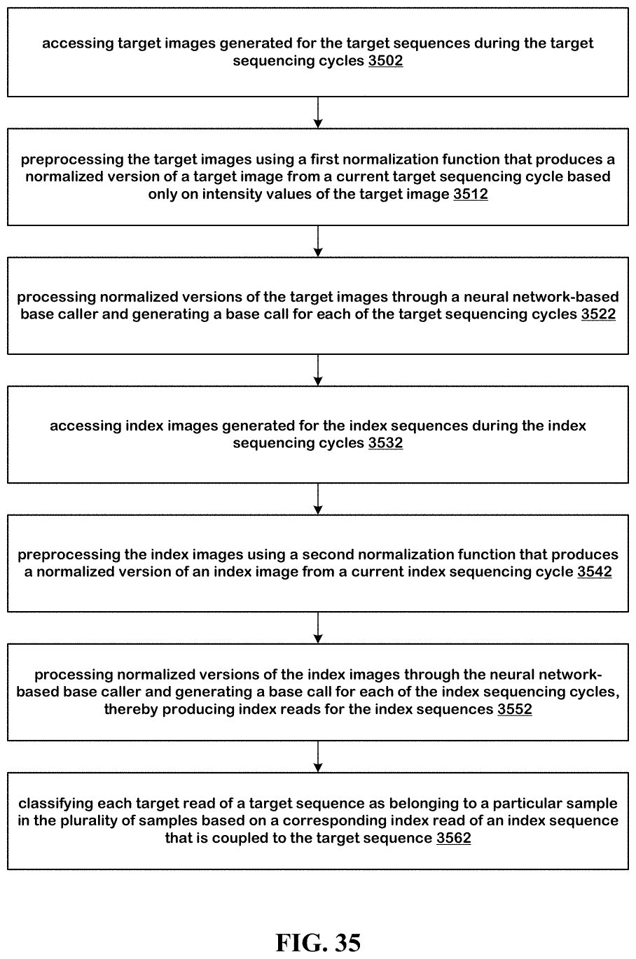

FIG. 35 is one implementation of a flow chart of an artificial intelligence-based method of base calling target sequences and index sequences. The target sequences are derived from a plurality of samples and coupled to the index sequences to form target-index sequences. Each index sequence is uniquely associated with a respective sample in the plurality of samples. The target-index sequences are pooled for sequencing during a sequencing run. The target sequences are sequenced during target sequencing cycles of the sequencing run and the index sequences are sequenced during index sequencing cycles of the sequencing run.

The method includes, at action 3502, accessing target images generated for the target sequences during the target sequencing cycles. The target images depict intensity emissions generated as a result of nucleotide incorporation in the target sequences.

The method further includes, at action 3512, preprocessing the target images using a first normalization function that produces a normalized version of a target image from a current target sequencing cycle based only on intensity values of the target image.

The method further includes, at action 3522, processing normalized versions of the target images through a neural network-based base caller and generating a base call for each of the target sequencing cycles, thereby producing target reads for the target sequences.

The method further includes, at action 3532, accessing index images generated for the index sequences during the index sequencing cycles. The index images depict intensity emissions generated as a result of nucleotide incorporation in the index sequences.

The method further includes, at action 3542, preprocessing the index images using a second normalization function that produces a normalized version of an index image from a current index sequencing cycle based on (i) intensity values of index images from one or more preceding index sequencing cycles, (ii) intensity values of index images from one or more succeeding index sequencing cycles, and (iii) intensity values of index images from the current index sequencing cycle.

The method further includes, at action 3552, processing normalized versions of the index images through the neural network-based base caller and generating a base call for each of the index sequencing cycles, thereby producing index reads for the index sequences.

The method further includes, at action 3562, classifying each target read of a target sequence as belonging to a particular sample in the plurality of samples based on a corresponding index read of an index sequence that is coupled to the target sequence.

Each of the features discussed in the particular implementation section for other implementations apply equally to this implementation. As indicated above, all the other features are not repeated here and should be considered repeated by reference. The reader will understand how features identified in these implementations can readily be combined with sets of base features identified in other implementations.

In one implementation, the first normalization function calculates a lower percentile of the intensity values of the target image, and an upper percentile of the intensity values of the target image, such that, in the normalized version of the target image, a first percentage of normalized intensity values are below the lower percentile, a second percentage of the normalized intensity values are above the upper percentile, and a third percentage of the normalized intensity values are between the lower and upper percentiles.

In one implementation, the second normalization function calculates a lower percentile of (i) the intensity values of the index images from the one or more preceding index sequencing cycles, (ii) the intensity values of the index images from the one or more succeeding index sequencing cycles, and (iii) the intensity values of the index images from the current index sequencing cycle, and an upper percentile of (i) the intensity values of the index images from the one or more preceding index sequencing cycles, (ii) the intensity values of the index images from the one or more succeeding index sequencing cycles, and (iii) the intensity values of the index images from the current index sequencing cycle, such that, in the normalized version of the index image, a first percentage of normalized intensity values are below the lower percentile, a second percentage of the normalized intensity values are above the upper percentile, and a third percentage of the normalized intensity values are between the lower and upper percentiles.

Other implementations of the method described in this section can include a non-transitory computer readable storage medium storing instructions executable by a processor to perform any of the methods described above. Yet another implementation of the method described in this section can include a system including memory and one or more processors operable to execute instructions, stored in the memory, to perform any of the methods described above.

The implementations disclosed herein may be implemented as a method, apparatus, system, or article of manufacture using standard programming or engineering techniques to produce software, firmware, hardware, or any combination thereof. The term “article of manufacture” as used herein refers to code or logic implemented in hardware or computer readable media such as optical storage devices, and volatile or non-volatile memory devices. Such hardware may include, but is not limited to, field programmable gate arrays (FPGAs), application-specific integrated circuits (ASICs), complex programmable logic devices (CPLDs), programmable logic arrays (PLAs), microprocessors, or other similar processing devices. In particular implementations, information or algorithms set forth herein are present in non-transient storage media.

One or more implementations of the technology disclosed, or elements thereof can be implemented in the form of a computer product including a non-transitory computer readable storage medium with computer usable program code for performing the method steps indicated. Furthermore, one or more implementations of the technology disclosed, or elements thereof can be implemented in the form of an apparatus including a memory and at least one processor that is coupled to the memory and operative to perform exemplary method steps. Yet further, in another aspect, one or more implementations of the technology disclosed or elements thereof can be implemented in the form of means for carrying out one or more of the method steps described herein; the means can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media).

As used herein, the term “analyte” is intended to mean a point or area in a pattern that can be distinguished from other points or areas according to relative location. An individual analyte can include one or more molecules of a particular type. For example, an analyte can include a single target nucleic acid molecule having a particular sequence or an analyte can include several nucleic acid molecules having the same sequence (and/or complementary sequence, thereof). Different molecules that are at different analytes of a pattern can be differentiated from each other according to the locations of the analytes in the pattern. Example analytes include without limitation, wells in a substrate, beads (or other particles) in or on a substrate, projections from a substrate, ridges on a substrate, pads of gel material on a substrate, or channels in a substrate.

Any of a variety of target analytes that are to be detected, characterized, or identified can be used in an apparatus, system or method set forth herein. Exemplary analytes include, but are not limited to, nucleic acids (e.g., DNA, RNA or analogs thereof), proteins, polysaccharides, cells, antibodies, epitopes, receptors, ligands, enzymes (e.g., kinases, phosphatases or polymerases), small molecule drug candidates, cells, viruses, organisms, or the like.

The terms “analyte,” “nucleic acid,” “nucleic acid molecule,” and “polynucleotide” are used interchangeably herein. In various implementations, nucleic acids may be used as templates as provided herein (e.g., a nucleic acid template, or a nucleic acid complement that is complementary to a nucleic acid nucleic acid template) for particular types of nucleic acid analysis, including but not limited to nucleic acid amplification, nucleic acid expression analysis, and/or nucleic acid sequence determination or suitable combinations thereof. Nucleic acids in certain implementations include, for instance, linear polymers of deoxyribonucleotides in 3′-5′ phosphodiester or other linkages, such as deoxyribonucleic acids (DNA), for example, single- and double-stranded DNA, genomic DNA, copy DNA or complementary DNA (cDNA), recombinant DNA, or any form of synthetic or modified DNA. In other implementations, nucleic acids include for instance, linear polymers of ribonucleotides in 3′-5′ phosphodiester or other linkages such as ribonucleic acids (RNA), for example, single- and double-stranded RNA, messenger (mRNA), copy RNA or complementary RNA (cRNA), alternatively spliced mRNA, ribosomal RNA, small nucleolar RNA (snoRNA), microRNAs (miRNA), small interfering RNAs (sRNA), piwi RNAs (piRNA), or any form of synthetic or modified RNA. Nucleic acids used in the compositions and methods of the present invention may vary in length and may be intact or full-length molecules or fragments or smaller parts of larger nucleic acid molecules. In particular implementations, a nucleic acid may have one or more detectable labels, as described elsewhere herein.

The terms “analyte,” “cluster,” “nucleic acid cluster,” “nucleic acid colony,” and “DNA cluster” are used interchangeably and refer to a plurality of copies of a nucleic acid template and/or complements thereof attached to a solid support. Typically and in certain preferred implementations, the nucleic acid cluster comprises a plurality of copies of template nucleic acid and/or complements thereof, attached via their 5′ termini to the solid support. The copies of nucleic acid strands making up the nucleic acid clusters may be in a single or double stranded form. Copies of a nucleic acid template that are present in a cluster can have nucleotides at corresponding positions that differ from each other, for example, due to presence of a label moiety. The corresponding positions can also contain analog structures having different chemical structure but similar Watson-Crick base-pairing properties, such as is the case for uracil and thymine.

Colonies of nucleic acids can also be referred to as “nucleic acid clusters.” Nucleic acid colonies can optionally be created by cluster amplification or bridge amplification techniques as set forth in further detail elsewhere herein. Multiple repeats of a target sequence can be present in a single nucleic acid molecule, such as a concatemer created using a rolling circle amplification procedure.

The nucleic acid clusters of the invention can have different shapes, sizes and densities depending on the conditions used. For example, clusters can have a shape that is substantially round, multi-sided, donut-shaped or ring-shaped. The diameter of a nucleic acid cluster can be designed to be from about 0.2 μm to about 6 μm, about 0.3 μm to about 4 μm, about 0.4 μm to about 3 μm, about 0.5 μm to about 2 μm, about 0.75 μm to about 1.5 μm, or any intervening diameter. In a particular implementation, the diameter of a nucleic acid cluster is about 0.5 μm, about 1 μm, about 1.5 μm, about 2 μm, about 2.5 μm, about 3 μm, about 4 μm, about 5 μm, or about 6 μm. The diameter of a nucleic acid cluster may be influenced by a number of parameters, including, but not limited to the number of amplification cycles performed in producing the cluster, the length of the nucleic acid template or the density of primers attached to the surface upon which clusters are formed. The density of nucleic acid clusters can be designed to typically be in the range of 0.1/mm², 1/mm², 10/mm², 100/mm², 1,000/mm², 10,000/mm² to 100,000/mm². The present invention further contemplates, in part, higher density nucleic acid clusters, for example, 100,000/mm² to 1,000,000/mm² and 1,000,000/mm² to 10,000,000/mm².

As used herein, an “analyte” is an area of interest within a specimen or field of view. When used in connection with microarray devices or other molecular analytical devices, an analyte refers to the area occupied by similar or identical molecules. For example, an analyte can be an amplified oligonucleotide or any other group of a polynucleotide or polypeptide with a same or similar sequence. In other implementations, an analyte can be any element or group of elements that occupy a physical area on a specimen. For example, an analyte could be a parcel of land, a body of water or the like. When an analyte is imaged, each analyte will have some area. Thus, in many implementations, an analyte is not merely one pixel.

The distances between analytes can be described in any number of ways. In some implementations, the distances between analytes can be described from the center of one analyte to the center of another analyte. In other implementations, the distances can be described from the edge of one analyte to the edge of another analyte, or between the outer-most identifiable points of each analyte. The edge of an analyte can be described as the theoretical or actual physical boundary on a chip, or some point inside the boundary of the analyte. In other implementations, the distances can be described in relation to a fixed point on the specimen or in the image of the specimen.

### Clauses

The following clauses are part of this disclosure:

### Index Reads

1. An artificial intelligence-based method of base calling index sequences, the method including:

accessing index images generated for the index sequences during index sequencing cycles of a sequencing run, wherein the index images depict intensity emissions generated as a result of nucleotide incorporation in the index sequences during the sequencing run;

preprocessing the index images using a normalization function that produces a normalized version of an index image from a current index sequencing cycle based on
(i) intensity values of index images from one or more preceding index sequencing cycles,
(ii) intensity values of index images from one or more succeeding index sequencing cycles, and
(iii) intensity values of index images from the current index sequencing cycle; and processing normalized versions of the index images through a neural network-based base caller and generating a base call for each of the index sequencing cycles, thereby producing index reads for the index sequences.
   2. The artificial intelligence-based method of clause 1, wherein the normalization function calculates:

a lower percentile of (i) the intensity values of the index images from the one or more preceding index sequencing cycles, (ii) the intensity values of the index images from the one or more succeeding index sequencing cycles, and (iii) the intensity values of the index images from the current index sequencing cycle, and

an upper percentile of (i) the intensity values of the index images from the one or more preceding index sequencing cycles, (ii) the intensity values of the index images from the one or more succeeding index sequencing cycles, and (iii) the intensity values of the index images from the current index sequencing cycle, such that,
in the normalized version of the index image,
   a first percentage of normalized intensity values are below the lower percentile,
   a second percentage of the normalized intensity values are above the upper percentile, and
   a third percentage of the normalized intensity values are between the lower and upper percentiles.
      3. The artificial intelligence-based method of clause 1, wherein,

taken together, nucleotides depicted by the index images from the current, preceding, and succeeding index sequencing cycles
are cumulatively more diverse than

nucleotides depicted only by the index images from the current index sequencing cycle.

4. The artificial intelligence-based method of clause 3, wherein at least one index image in the index images from the preceding and succeeding index sequencing cycles depicts one or more nucleotides in a detectable signal state.
5. The artificial intelligence-based method of clause 3, wherein the nucleotides depicted by the index images from the current index sequencing cycle are low-complexity patterns in which some of four bases A, C, T, and G are represented at a frequency of less than 15%, 10%, or 5% of all the nucleotides.
6. The artificial intelligence-based method of clause 5, wherein, taken together, the nucleotides depicted by the index images from the current, preceding, and succeeding index sequencing cycles cumulatively form high-complexity patterns in which each of the four bases A, C, T, and G is represented at a frequency of at least 20%, 25%, or 30% of all the nucleotides.
7. The artificial intelligence-based method of clause 1, further including:

preprocessing the index images using the normalization function during training of the neural network-based base caller as well as during inference.

8. The artificial intelligence-based method of clause 1, further including:

preprocessing the index images using an augmentation function that produces an augmented version of an index image by multiplying intensity values of the index image with a scaling factor and adding an offset value to the multiplication's result; and processing augmented versions of the index images through the neural network-based base caller and generating a base call for each of the index sequencing cycles, thereby producing index reads for the index sequences.

9. The artificial intelligence-based method of clause 8, further including:

preprocessing the index images using the augmentation function only during the training of the neural network-based base caller and not during the inference.

10. The artificial intelligence-based method of clause 1, further including:

preprocessing the index images using the normalization function that produces the normalized version of the index image from the current index sequencing cycle based on
(i) intensity values of index images from one or more non-current index sequencing cycles, and
(ii) intensity values of index images from the current index sequencing cycle.
   11. The artificial intelligence-based method of clause 10, wherein the non-current index sequencing cycles comprise initial index sequencing cycles of the sequencing.
   12. The artificial intelligence-based method of clause 10, wherein the non-current index sequencing cycles comprise intermediate index sequencing cycles of the sequencing.
   13. The artificial intelligence-based method of clause 10, wherein the non-current index sequencing cycles comprise terminal index sequencing cycles of the sequencing.
   14. The artificial intelligence-based method of clause 13, wherein the non-current index sequencing cycles comprise a combination of the initial index sequencing cycles, the intermediate index sequencing cycles, and the terminal index sequencing cycles.
   15. The artificial intelligence-based method of clause 10, wherein at least one index image from the non-current index sequencing cycles depicts one or more nucleotides in the detectable signal state.
   16. An artificial intelligence-based method of base calling analytes at index sequencing cycles of a sequencing run, the method including:

preprocessing index images generated during the index sequencing cycles using a normalization function that produces a normalized version of an index image from a current index sequencing cycle based on
(i) intensity values of index images from one or more preceding index sequencing cycles,
(ii) intensity values of index images from one or more succeeding index sequencing cycles, and
(iii) intensity values of index images from the current index sequencing cycle; for a particular analyte being base called at the current index sequencing cycle,
extracting index image patches from normalized versions of the index images from the current, preceding, succeeding index sequencing cycles, such that,
   each normalized index image patch depicts intensity emissions of the particular analyte, of some adjacent analytes, and of their surrounding background generated as a result of nucleotide incorporation in corresponding index sequences of the particular analyte and the adjacent analytes during the current index sequencing cycle;

convolving the normalized index image patches through a convolutional neural network and generating a convolved representation; and

base calling the particular analyte at the current index sequencing cycle based on the convolved representation.

17. An artificial intelligence-based method of base calling target sequences and index sequences, wherein the target sequences are derived from a plurality of samples and coupled to the index sequences to form target-index sequences, wherein each index sequence is uniquely associated with a respective sample in the plurality of samples, wherein the target-index sequences are pooled for sequencing during a sequencing run, and wherein the target sequences are sequenced during target sequencing cycles of the sequencing run and the index sequences are sequenced during index sequencing cycles of the sequencing run, the method including:

accessing target images generated for the target sequences during the target sequencing cycles, wherein the target images depict intensity emissions generated as a result of nucleotide incorporation in the target sequences;

preprocessing the target images using a first normalization function that produces a normalized version of a target image from a current target sequencing cycle based only on intensity values of the target image;

processing normalized versions of the target images through a neural network-based base caller and generating a base call for each of the target sequencing cycles, thereby producing target reads for the target sequences;

accessing index images generated for the index sequences during the index sequencing cycles, wherein the index images depict intensity emissions generated as a result of nucleotide incorporation in the index sequences;

preprocessing the index images using a second normalization function that produces a normalized version of an index image from a current index sequencing cycle based on
(i) intensity values of index images from one or more preceding index sequencing cycles,
(ii) intensity values of index images from one or more succeeding index sequencing cycles, and
(iii) intensity values of index images from the current index sequencing cycle;

processing normalized versions of the index images through the neural network-based base caller and generating a base call for each of the index sequencing cycles, thereby producing index reads for the index sequences; and

classifying each target read of a target sequence as belonging to a particular sample in the plurality of samples based on a corresponding index read of an index sequence that is coupled to the target sequence.

18. The artificial intelligence-based method of clause 17, wherein the first normalization function calculates:

a lower percentile of the intensity values of the target image, and

an upper percentile of the intensity values of the target image, such that,
in the normalized version of the target image,
   a first percentage of normalized intensity values are below the lower percentile,
   a second percentage of the normalized intensity values are above the upper percentile, and
   a third percentage of the normalized intensity values are between the lower and upper percentiles.
      19. The artificial intelligence-based method of clause 17, wherein the second normalization function calculates:

a lower percentile of (i) the intensity values of the index images from the one or more preceding index sequencing cycles, (ii) the intensity values of the index images from the one or more succeeding index sequencing cycles, and (iii) the intensity values of the index images from the current index sequencing cycle, and

an upper percentile of (i) the intensity values of the index images from the one or more preceding index sequencing cycles, (ii) the intensity values of the index images from the one or more succeeding index sequencing cycles, and (iii) the intensity values of the index images from the current index sequencing cycle, such that,
in the normalized version of the index image,
   a first percentage of normalized intensity values are below the lower percentile,
   a second percentage of the normalized intensity values are above the upper percentile, and
   a third percentage of the normalized intensity values are between the lower and upper percentiles.

### Index and Normal Reads

20. An artificial intelligence-based method of base calling target sequences and index sequences, wherein the target sequences are derived from a plurality of samples and coupled to the index sequences to form target-index sequences, wherein each index sequence is uniquely associated with a respective sample in the plurality of samples, wherein the target-index sequences are pooled for sequencing during a sequencing run, and wherein the target sequences are sequenced during target sequencing cycles of the sequencing run and the index sequences are sequenced during index sequencing cycles of the sequencing run, the method including:

accessing target images generated for the target sequences during the target sequencing cycles, wherein the target images depict intensity emissions generated as a result of nucleotide incorporation in the target sequences;

preprocessing the target images using a normalization function that produces a normalized version of a target image from a current target sequencing cycle based on (i) intensity values of target images from one or more preceding target sequencing cycles, (ii) intensity values of target images from one or more succeeding target sequencing cycles, and (iii) intensity values of target images from the current target sequencing cycle;

accessing index images generated for the index sequences during the index sequencing cycles, wherein the index images depict intensity emissions generated as a result of nucleotide incorporation in the index sequences;

preprocessing the index images using the normalization function that produces a normalized version of an index image from a current index sequencing cycle based on (i) intensity values of index images from one or more preceding index sequencing cycles, (ii) intensity values of index images from one or more succeeding index sequencing cycles, and (iii) intensity values of index images from the current index sequencing cycle;

processing normalized versions of the target images through a neural network-based base caller and generating a base call for each of the target sequencing cycles, thereby producing target reads for the target sequences;

processing normalized versions of the index images through the neural network-based base caller and generating a base call for each of the index sequencing cycles, thereby producing index reads for the index sequences; and

classifying each target read of a target sequence as belonging to a particular sample in the plurality of samples based on a corresponding index read of an index sequence that is coupled to the target sequence.

21. The artificial intelligence-based method of clause 20, wherein the normalization function calculates:

a lower percentile of (i) the intensity values of the target images from the one or more preceding target sequencing cycles, (ii) the intensity values of the target images from the one or more succeeding target sequencing cycles, and (iii) the intensity values of the target images from the current target sequencing cycle, and

an upper percentile of (i) the intensity values of the target images from the one or more preceding target sequencing cycles, (ii) the intensity values of the target images from the one or more succeeding target sequencing cycles, and (iii) the intensity values of the target images from the current target sequencing cycle, such that,
in the normalized version of the target image,
   a first percentage of normalized intensity values are below the lower percentile,
   a second percentage of the normalized intensity values are above the upper percentile, and
   a third percentage of the normalized intensity values are between the lower and upper percentiles.
      22. The artificial intelligence-based method of clause 20, wherein the normalization function calculates:

a lower percentile of (i) the intensity values of the index images from the one or more preceding index sequencing cycles, (ii) the intensity values of the index images from the one or more succeeding index sequencing cycles, and (iii) the intensity values of the index images from the current index sequencing cycle, and

an upper percentile of (i) the intensity values of the index images from the one or more preceding index sequencing cycles, (ii) the intensity values of the index images from the one or more succeeding index sequencing cycles, and (iii) the intensity values of the index images from the current index sequencing cycle, such that,
in the normalized version of the index image,
   a first percentage of normalized intensity values are below the lower percentile,
   a second percentage of the normalized intensity values are above the upper percentile, and
   a third percentage of the normalized intensity values are between the lower and upper percentiles.
      23. The artificial intelligence-based method of clause 20, further including:

preprocessing the target images and the index images using the normalization function during training of the neural network-based base caller as well as during inference.

24. The artificial intelligence-based method of clause 20, further including:

preprocessing the target images using an augmentation function that produces an augmented version of a target image by multiplying intensity values of the target image with a scaling factor and adding an offset value to the multiplication's result; and

processing augmented versions of the target images through the neural network-based base caller and generating a base call for each of the target sequencing cycles, thereby producing target reads for the target sequences.

25. The artificial intelligence-based method of clause 20, further including:

preprocessing the index images using the augmentation function that produces an augmented version of an index image by multiplying intensity values of the index image with a scaling factor and adding an offset value to the multiplication's result; and

processing augmented versions of the index images through the neural network-based base caller and generating a base call for each of the index sequencing cycles, thereby producing index reads for the index sequences.

26. The artificial intelligence-based method of clause 20, further including:

preprocessing the target images and the index images using the augmentation function only during the training of the neural network-based base caller and not during the inference.

27. An artificial intelligence-based method of base calling sequences, the method including:

accessing target images generated for target sequences during target sequencing cycles a sequencing run, wherein the target images depict intensity emissions generated as a result of nucleotide incorporation in the target sequences;

preprocessing the target images using a normalization function that produces a normalized version of a target image from a current target sequencing cycle based on (i) intensity values of target images from one or more preceding target sequencing cycles, (ii) intensity values of target images from one or more succeeding target sequencing cycles, and (iii) intensity values of target images from the current target sequencing cycle;

accessing index images generated for index sequences during index sequencing cycles of the sequencing run, wherein the index images depict intensity emissions generated as a result of nucleotide incorporation in the index sequences during the sequencing run;

preprocessing the index images using the normalization function that produces a normalized version of an index image from a current index sequencing cycle based on (i) intensity values of index images from one or more preceding index sequencing cycles, (ii) intensity values of index images from one or more succeeding index sequencing cycles, and (iii) intensity values of index images from the current index sequencing cycle;

processing normalized versions of the target images through a neural network-based base caller and generating a base call for each of the target sequencing cycles, thereby producing target reads for the target sequences; and

processing normalized versions of the index images through the neural network-based base caller and generating a base call for each of the index sequencing cycles, thereby producing index reads for the index sequences.

Other implementations of the method described above can include a non-transitory computer readable storage medium storing instructions executable by a processor to perform any of the methods described above. Yet another implementation of the method described in this section can include a system including memory and one or more processors operable to execute instructions, stored in the memory, to perform any of the methods described above.

28. An artificial intelligence-based method of base calling sequences, the method including:

accessing target images generated for target sequences during target sequencing cycles a sequencing run, wherein the target images depict intensity emissions generated as a result of nucleotide incorporation in the target sequences;

accessing index images generated for index sequences during index sequencing cycles of the sequencing run, wherein the index images depict intensity emissions generated as a result of nucleotide incorporation in the index sequences during the sequencing run;

processing the target images through a neural network-based base caller and generating a base call for each of the target sequencing cycles, thereby producing target reads for the target sequences; and

processing the index images through the neural network-based base caller and generating a base call for each of the index sequencing cycles, thereby producing index reads for the index sequences.

29. A system including one or more processors coupled to memory, the memory loaded with computer instructions to base call index sequences, the instructions, when executed on the processors, implement actions comprising:

accessing index images generated for the index sequences during index sequencing cycles of a sequencing run, wherein the index images depict intensity emissions generated as a result of nucleotide incorporation in the index sequences during the sequencing run;

preprocessing the index images using a normalization function that produces a normalized version of an index image from a current index sequencing cycle based on
(i) intensity values of index images from one or more preceding index sequencing cycles,
(ii) intensity values of index images from one or more succeeding index sequencing cycles, and
(iii) intensity values of index images from the current index sequencing cycle; and

processing normalized versions of the index images through a neural network-based base caller and generating a base call for each of the index sequencing cycles, thereby producing index reads for the index sequences.

30. The system of clause 29, implementing each of the clauses which ultimately depend from clauses 1, 16, 17, 20, and 27.
31. A system including one or more processors coupled to memory, the memory loaded with computer instructions to base call analytes at index sequencing cycles of a sequencing run, the instructions, when executed on the processors, implement actions comprising:

preprocessing index images generated during the index sequencing cycles using a normalization function that produces a normalized version of an index image from a current index sequencing cycle based on
(i) intensity values of index images from one or more preceding index sequencing cycles,
(ii) intensity values of index images from one or more succeeding index sequencing cycles, and
(iii) intensity values of index images from the current index sequencing cycle;

for a particular analyte being base called at the current index sequencing cycle,
extracting index image patches from normalized versions of the index images from the current, preceding, succeeding index sequencing cycles, such that,
   each normalized index image patch depicts intensity emissions of the particular analyte, of some adjacent analytes, and of their surrounding background generated as a result of nucleotide incorporation in corresponding index sequences of the particular analyte and the adjacent analytes during the current index sequencing cycle;

convolving the normalized index image patches through a convolutional neural network and generating a convolved representation; and

base calling the particular analyte at the current index sequencing cycle based on the convolved representation.

32. The system of clause 31, implementing each of the clauses which ultimately depend from clauses 1, 16, 17, 20, and 27.
33. A system including one or more processors coupled to memory, the memory loaded with computer instructions to base call target sequences and index sequences, wherein the target sequences are derived from a plurality of samples and coupled to the index sequences to form target-index sequences, wherein each index sequence is uniquely associated with a respective sample in the plurality of samples, wherein the target-index sequences are pooled for sequencing during a sequencing run, and wherein the target sequences are sequenced during target sequencing cycles of the sequencing run and the index sequences are sequenced during index sequencing cycles of the sequencing run, the instructions, when executed on the processors, implement actions comprising:

accessing target images generated for the target sequences during the target sequencing cycles, wherein the target images depict intensity emissions generated as a result of nucleotide incorporation in the target sequences;

preprocessing the target images using a first normalization function that produces a normalized version of a target image from a current target sequencing cycle based only on intensity values of the target image;

processing normalized versions of the target images through a neural network-based base caller and generating a base call for each of the target sequencing cycles, thereby producing target reads for the target sequences;

accessing index images generated for the index sequences during the index sequencing cycles, wherein the index images depict intensity emissions generated as a result of nucleotide incorporation in the index sequences;

preprocessing the index images using a second normalization function that produces a normalized version of an index image from a current index sequencing cycle based on
(i) intensity values of index images from one or more preceding index sequencing cycles,
(ii) intensity values of index images from one or more succeeding index sequencing cycles, and
(iii) intensity values of index images from the current index sequencing cycle;

processing normalized versions of the index images through the neural network-based base caller and generating a base call for each of the index sequencing cycles, thereby producing index reads for the index sequences; and

classifying each target read of a target sequence as belonging to a particular sample in the plurality of samples based on a corresponding index read of an index sequence that is coupled to the target sequence.

34. The system of clause 33, implementing each of the clauses which ultimately depend from clauses 1, 16, 17, 20, and 27.
35. A system including one or more processors coupled to memory, the memory loaded with computer instructions to base call target sequences and index sequences, wherein the target sequences are derived from a plurality of samples and coupled to the index sequences to form target-index sequences, wherein each index sequence is uniquely associated with a respective sample in the plurality of samples, wherein the target-index sequences are pooled for sequencing during a sequencing run, and wherein the target sequences are sequenced during target sequencing cycles of the sequencing run and the index sequences are sequenced during index sequencing cycles of the sequencing run, the instructions, when executed on the processors, implement actions comprising:

accessing target images generated for the target sequences during the target sequencing cycles, wherein the target images depict intensity emissions generated as a result of nucleotide incorporation in the target sequences;

preprocessing the target images using a normalization function that produces a normalized version of a target image from a current target sequencing cycle based on (i) intensity values of target images from one or more preceding target sequencing cycles, (ii) intensity values of target images from one or more succeeding target sequencing cycles, and (iii) intensity values of target images from the current target sequencing cycle;

accessing index images generated for the index sequences during the index sequencing cycles, wherein the index images depict intensity emissions generated as a result of nucleotide incorporation in the index sequences;

preprocessing the index images using the normalization function that produces a normalized version of an index image from a current index sequencing cycle based on (i) intensity values of index images from one or more preceding index sequencing cycles, (ii) intensity values of index images from one or more succeeding index sequencing cycles, and (iii) intensity values of index images from the current index sequencing cycle;

processing normalized versions of the target images through a neural network-based base caller and generating a base call for each of the target sequencing cycles, thereby producing target reads for the target sequences;

processing normalized versions of the index images through the neural network-based base caller and generating a base call for each of the index sequencing cycles, thereby producing index reads for the index sequences; and classifying each target read of a target sequence as belonging to a particular sample in the plurality of samples based on a corresponding index read of an index sequence that is coupled to the target sequence.

36. The system of clause 35, implementing each of the clauses which ultimately depend from clauses 1, 16, 17, 20, and 27.
37. A system including one or more processors coupled to memory, the memory loaded with computer instructions to base call sequences, the instructions, when executed on the processors, implement actions comprising:

accessing target images generated for target sequences during target sequencing cycles a sequencing run, wherein the target images depict intensity emissions generated as a result of nucleotide incorporation in the target sequences;

preprocessing the target images using a normalization function that produces a normalized version of a target image from a current target sequencing cycle based on (i) intensity values of target images from one or more preceding target sequencing cycles, (ii) intensity values of target images from one or more succeeding target sequencing cycles, and (iii) intensity values of target images from the current target sequencing cycle;

accessing index images generated for index sequences during index sequencing cycles of the sequencing run, wherein the index images depict intensity emissions generated as a result of nucleotide incorporation in the index sequences during the sequencing run;

preprocessing the index images using the normalization function that produces a normalized version of an index image from a current index sequencing cycle based on (i) intensity values of index images from one or more preceding index sequencing cycles, (ii) intensity values of index images from one or more succeeding index sequencing cycles, and (iii) intensity values of index images from the current index sequencing cycle;

processing normalized versions of the target images through a neural network-based base caller and generating a base call for each of the target sequencing cycles, thereby producing target reads for the target sequences; and

processing normalized versions of the index images through the neural network-based base caller and generating a base call for each of the index sequencing cycles, thereby producing index reads for the index sequences.

38. The system of clause 37, implementing each of the clauses which ultimately depend from clauses 1, 16, 17, 20, and 27.
39. A system including one or more processors coupled to memory, the memory loaded with computer instructions to base call sequences, the instructions, when executed on the processors, implement actions comprising:

accessing target images generated for target sequences during target sequencing cycles a sequencing run, wherein the target images depict intensity emissions generated as a result of nucleotide incorporation in the target sequences;

accessing index images generated for index sequences during index sequencing cycles of the sequencing run, wherein the index images depict intensity emissions generated as a result of nucleotide incorporation in the index sequences during the sequencing run;

processing the target images through a neural network-based base caller and generating a base call for each of the target sequencing cycles, thereby producing target reads for the target sequences; and

processing the index images through the neural network-based base caller and generating a base call for each of the index sequencing cycles, thereby producing index reads for the index sequences.

40. The system of clause 39, implementing each of the clauses which ultimately depend from clauses 1, 16, 17, 20, and 27.
41. A non-transitory computer readable storage medium impressed with computer program instructions to base call index sequences, the instructions, when executed on a processor, implement a method comprising:

accessing index images generated for the index sequences during index sequencing cycles of a sequencing run, wherein the index images depict intensity emissions generated as a result of nucleotide incorporation in the index sequences during the sequencing run;

preprocessing the index images using a normalization function that produces a normalized version of an index image from a current index sequencing cycle based on
(i) intensity values of index images from one or more preceding index sequencing cycles,
(ii) intensity values of index images from one or more succeeding index sequencing cycles, and
(iii) intensity values of index images from the current index sequencing cycle; and

processing normalized versions of the index images through a neural network-based base caller and generating a base call for each of the index sequencing cycles, thereby producing index reads for the index sequences.

42. The non-transitory computer readable storage medium of clause 41, implementing each of the clauses which ultimately depend from clauses 1, 16, 17, 20, and 27.
43. A non-transitory computer readable storage medium impressed with computer program instructions to base call analytes at index sequencing cycles of a sequencing run, the instructions, when executed on a processor, implement a method comprising:

preprocessing index images generated during the index sequencing cycles using a normalization function that produces a normalized version of an index image from a current index sequencing cycle based on
(i) intensity values of index images from one or more preceding index sequencing cycles,
(ii) intensity values of index images from one or more succeeding index sequencing cycles, and
(iii) intensity values of index images from the current index sequencing cycle;

for a particular analyte being base called at the current index sequencing cycle,
extracting index image patches from normalized versions of the index images from the current, preceding, succeeding index sequencing cycles, such that,
   each normalized index image patch depicts intensity emissions of the particular analyte, of some adjacent analytes, and of their surrounding background generated as a result of nucleotide incorporation in corresponding index sequences of the particular analyte and the adjacent analytes during the current index sequencing cycle;

convolving the normalized index image patches through a convolutional neural network and generating a convolved representation; and

base calling the particular analyte at the current index sequencing cycle based on the convolved representation.

44. The non-transitory computer readable storage medium of clause 43, implementing each of the clauses which ultimately depend from clauses 1, 16, 17, 20, and 27.
45. A non-transitory computer readable storage medium impressed with computer program instructions to base call target sequences and index sequences, wherein the target sequences are derived from a plurality of samples and coupled to the index sequences to form target-index sequences, wherein each index sequence is uniquely associated with a respective sample in the plurality of samples, wherein the target-index sequences are pooled for sequencing during a sequencing run, and wherein the target sequences are sequenced during target sequencing cycles of the sequencing run and the index sequences are sequenced during index sequencing cycles of the sequencing run, the instructions, when executed on a processor, implement a method comprising:

accessing target images generated for the target sequences during the target sequencing cycles, wherein the target images depict intensity emissions generated as a result of nucleotide incorporation in the target sequences;

preprocessing the target images using a first normalization function that produces a normalized version of a target image from a current target sequencing cycle based only on intensity values of the target image;

processing normalized versions of the target images through a neural network-based base caller and generating a base call for each of the target sequencing cycles, thereby producing target reads for the target sequences;

accessing index images generated for the index sequences during the index sequencing cycles, wherein the index images depict intensity emissions generated as a result of nucleotide incorporation in the index sequences;

preprocessing the index images using a second normalization function that produces a normalized version of an index image from a current index sequencing cycle based on
(i) intensity values of index images from one or more preceding index sequencing cycles,
(ii) intensity values of index images from one or more succeeding index sequencing cycles, and
(iii) intensity values of index images from the current index sequencing cycle;

processing normalized versions of the index images through the neural network-based base caller and generating a base call for each of the index sequencing cycles, thereby producing index reads for the index sequences; and

classifying each target read of a target sequence as belonging to a particular sample in the plurality of samples based on a corresponding index read of an index sequence that is coupled to the target sequence.

46. The non-transitory computer readable storage medium of clause 45, implementing each of the clauses which ultimately depend from clauses 1, 16, 17, 20, and 27.
47. A non-transitory computer readable storage medium impressed with computer program instructions to base call target sequences and index sequences, wherein the target sequences are derived from a plurality of samples and coupled to the index sequences to form target-index sequences, wherein each index sequence is uniquely associated with a respective sample in the plurality of samples, wherein the target-index sequences are pooled for sequencing during a sequencing run, and wherein the target sequences are sequenced during target sequencing cycles of the sequencing run and the index sequences are sequenced during index sequencing cycles of the sequencing run, the instructions, when executed on a processor, implement a method comprising:

accessing target images generated for the target sequences during the target sequencing cycles, wherein the target images depict intensity emissions generated as a result of nucleotide incorporation in the target sequences;

preprocessing the target images using a normalization function that produces a normalized version of a target image from a current target sequencing cycle based on (i) intensity values of target images from one or more preceding target sequencing cycles, (ii) intensity values of target images from one or more succeeding target sequencing cycles, and (iii) intensity values of target images from the current target sequencing cycle;

accessing index images generated for the index sequences during the index sequencing cycles, wherein the index images depict intensity emissions generated as a result of nucleotide incorporation in the index sequences;

preprocessing the index images using the normalization function that produces a normalized version of an index image from a current index sequencing cycle based on (i) intensity values of index images from one or more preceding index sequencing cycles, (ii) intensity values of index images from one or more succeeding index sequencing cycles, and (iii) intensity values of index images from the current index sequencing cycle;

processing normalized versions of the target images through a neural network-based base caller and generating a base call for each of the target sequencing cycles, thereby producing target reads for the target sequences;

processing normalized versions of the index images through the neural network-based base caller and generating a base call for each of the index sequencing cycles, thereby producing index reads for the index sequences; and

classifying each target read of a target sequence as belonging to a particular sample in the plurality of samples based on a corresponding index read of an index sequence that is coupled to the target sequence.

48. The non-transitory computer readable storage medium of clause 47, implementing each of the clauses which ultimately depend from clauses 1, 16, 17, 20, and 27.
49. A non-transitory computer readable storage medium impressed with computer program instructions to base call sequences, the instructions, when executed on a processor, implement a method comprising:

accessing target images generated for target sequences during target sequencing cycles a sequencing run, wherein the target images depict intensity emissions generated as a result of nucleotide incorporation in the target sequences;

preprocessing the target images using a normalization function that produces a normalized version of a target image from a current target sequencing cycle based on (i) intensity values of target images from one or more preceding target sequencing cycles, (ii) intensity values of target images from one or more succeeding target sequencing cycles, and (iii) intensity values of target images from the current target sequencing cycle;

accessing index images generated for index sequences during index sequencing cycles of the sequencing run, wherein the index images depict intensity emissions generated as a result of nucleotide incorporation in the index sequences during the sequencing run;

preprocessing the index images using the normalization function that produces a normalized version of an index image from a current index sequencing cycle based on (i) intensity values of index images from one or more preceding index sequencing cycles, (ii) intensity values of index images from one or more succeeding index sequencing cycles, and (iii) intensity values of index images from the current index sequencing cycle;

processing normalized versions of the target images through a neural network-based base caller and generating a base call for each of the target sequencing cycles, thereby producing target reads for the target sequences; and

processing normalized versions of the index images through the neural network-based base caller and generating a base call for each of the index sequencing cycles, thereby producing index reads for the index sequences.

50. The non-transitory computer readable storage medium of clause 49, implementing each of the clauses which ultimately depend from clauses 1, 16, 17, 20, and 27.
51. A non-transitory computer readable storage medium impressed with computer program instructions base call sequences, the instructions, when executed on a processor, implement a method comprising:

accessing target images generated for target sequences during target sequencing cycles a sequencing run, wherein the target images depict intensity emissions generated as a result of nucleotide incorporation in the target sequences;

accessing index images generated for index sequences during index sequencing cycles of the sequencing run, wherein the index images depict intensity emissions generated as a result of nucleotide incorporation in the index sequences during the sequencing run;

processing the target images through a neural network-based base caller and generating a base call for each of the target sequencing cycles, thereby producing target reads for the target sequences; and

processing the index images through the neural network-based base caller and generating a base call for each of the index sequencing cycles, thereby producing index reads for the index sequences.

52. The non-transitory computer readable storage medium of clause 51, implementing each of the clauses which ultimately depend from clauses 1, 16, 17, 20, and 27.

What is claimed is:

1. An artificial intelligence-based method of base calling index sequences, the method including:
accessing index images generated for the index sequences during index sequencing cycles of a sequencing run, wherein the index images depict intensity emissions generated as a result of nucleotide incorporation in the index sequences during the sequencing run;
preprocessing the index images using a normalization function that produces a normalized version of an index image from a current index sequencing cycle based on
(i) intensity values of index images from one or more preceding index sequencing cycles,
(ii) intensity values of index images from one or more succeeding index sequencing cycles, and
(iii) intensity values of index images from the current index sequencing cycle; and
processing normalized versions of the index images through a neural network-based base caller and generating a base call for each of the index sequencing cycles, thereby producing index reads for the index sequences.

2. The artificial intelligence-based method of claim 1, wherein the normalization function calculates:
a lower percentile of (i) the intensity values of the index images from the one or more preceding index sequencing cycles, (ii) the intensity values of the index images from the one or more succeeding index sequencing cycles, and (iii) the intensity values of the index images from the current index sequencing cycle, and
an upper percentile of (i) the intensity values of the index images from the one or more preceding index sequencing cycles, (ii) the intensity values of the index images from the one or more succeeding index sequencing cycles, and (iii) the intensity values of the index images from the current index sequencing cycle, such that,
in the normalized version of the index image,
a first percentage of normalized intensity values are below the lower percentile,
a second percentage of the normalized intensity values are above the upper percentile, and
a third percentage of the normalized intensity values are between the lower and upper percentiles.

3. The artificial intelligence-based method of claim 1, wherein,
taken together, nucleotides depicted by the index images from the current, preceding, and succeeding index sequencing cycles
are cumulatively more diverse than
nucleotides depicted only by the index images from the current index sequencing cycle.

4. The artificial intelligence-based method of claim 3, wherein at least one index image in the index images from the preceding and succeeding index sequencing cycles depicts one or more nucleotides in a detectable signal state.

5. The artificial intelligence-based method of claim 3, wherein the nucleotides depicted by the index images from the current index sequencing cycle are low-complexity patterns in which some of four bases A, C, T, and G are represented at a frequency of less than 15%, 10%, or 5% of all the nucleotides.

6. The artificial intelligence-based method of claim 5, wherein, taken together, the nucleotides depicted by the index images from the current, preceding, and succeeding index sequencing cycles cumulatively form high-complexity patterns in which each of the four bases A, C, T, and G are represented at a frequency of at least 20%, 25%, or 30% of all the nucleotides.

7. The artificial intelligence-based method of claim 1, further including:
preprocessing the index images using the normalization function during training of the neural network-based base caller as well as during inference.

8. The artificial intelligence-based method of claim 1, further including:
preprocessing the index images using an augmentation function that produces an augmented version of an index image by multiplying intensity values of the index image with a scaling factor and adding an offset value to the multiplication's result; and
processing augmented versions of the index images through the neural network-based base caller and generating a base call for each of the index sequencing cycles, thereby producing index reads for the index sequences.

9. The artificial intelligence-based method of claim 8, further including:
preprocessing the index images using the augmentation function only during the training of the neural network-based base caller and not during the inference.

10. The artificial intelligence-based method of claim 1, further including:
preprocessing the index images using the normalization function that produces the normalized version of the index image from the current index sequencing cycle based on
(i) intensity values of index images from one or more non-current index sequencing cycles, and
(ii) intensity values of index images from the current index sequencing cycle.

11. The artificial intelligence-based method of claim 10, wherein the non-current index sequencing cycles comprise initial index sequencing cycles of the sequencing.

12. The artificial intelligence-based method of claim 10, wherein the non-current index sequencing cycles comprise intermediate index sequencing cycles of the sequencing.

13. The artificial intelligence-based method of claim 10, wherein the non-current index sequencing cycles comprise terminal index sequencing cycles of the sequencing.

14. The artificial intelligence-based method of claim 13, wherein the non-current index sequencing cycles comprise a combination of the initial index sequencing cycles, the intermediate index sequencing cycles, and the terminal index sequencing cycles.

15. The artificial intelligence-based method of claim 10, wherein at least one index image from the non-current index sequencing cycles depicts one or more nucleotides in the detectable signal state.

16. An artificial intelligence-based method of base calling analytes at index sequencing cycles of a sequencing run, the method including:
preprocessing index images generated during the index sequencing cycles using a normalization function that produces a normalized version of an index image from a current index sequencing cycle based on
(i) intensity values of index images from one or more preceding index sequencing cycles,
(ii) intensity values of index images from one or more succeeding index sequencing cycles, and
(iii) intensity values of index images from the current index sequencing cycle;
for a particular analyte being base called at the current index sequencing cycle,
extracting index image patches from normalized versions of the index images from the current, preceding, succeeding index sequencing cycles, such that,
each normalized index image patch depicts intensity emissions of the particular analyte, of some adjacent analytes, and of their surrounding background generated as a result of nucleotide incorporation in corresponding index sequences of the particular analyte and the adjacent analytes during the current index sequencing cycle;
convolving the normalized index image patches through a convolutional neural network and generating a convolved representation; and
base calling the particular analyte at the current index sequencing cycle based on the convolved representation.

17. An artificial intelligence-based method of base calling target sequences and index sequences, wherein the target sequences are derived from a plurality of samples and coupled to the index sequences to form target-index sequences, wherein each index sequence is uniquely associated with a respective sample in the plurality of samples, wherein the target-index sequences are pooled for sequencing during a sequencing run, and wherein the target sequences are sequenced during target sequencing cycles of the sequencing run and the index sequences are sequenced during index sequencing cycles of the sequencing run, the method including:
accessing target images generated for the target sequences during the target sequencing cycles, wherein the target images depict intensity emissions generated as a result of nucleotide incorporation in the target sequences;
preprocessing the target images using a first normalization function that produces a normalized version of a target image from a current target sequencing cycle based only on intensity values of the target image;
processing normalized versions of the target images through a neural network-based base caller and generating a base call for each of the target sequencing cycles, thereby producing target reads for the target sequences;
accessing index images generated for the index sequences during the index sequencing cycles, wherein the index images depict intensity emissions generated as a result of nucleotide incorporation in the index sequences;
preprocessing the index images using a second normalization function that produces a normalized version of an index image from a current index sequencing cycle based on
(i) intensity values of index images from one or more preceding index sequencing cycles,
(ii) intensity values of index images from one or more succeeding index sequencing cycles, and
(iii) intensity values of index images from the current index sequencing cycle;
processing normalized versions of the index images through the neural network-based base caller and generating a base call for each of the index sequencing cycles, thereby producing index reads for the index sequences; and
classifying each target read of a target sequence as belonging to a particular sample in the plurality of samples based on a corresponding index read of an index sequence that is coupled to the target sequence.

18. The artificial intelligence-based method of claim 17, wherein the first normalization function calculates
a lower percentile of the intensity values of the target image, and
an upper percentile of the intensity values of the target image, such that,
in the normalized version of the target image,
a first percentage of normalized intensity values are below the lower percentile,
a second percentage of the normalized intensity values are above the upper percentile, and
a third percentage of the normalized intensity values are between the lower and upper percentiles.

19. An artificial intelligence-based method of base calling target sequences and index sequences, wherein the target sequences are derived from a plurality of samples and coupled to the index sequences to form target-index sequences, wherein each index sequence is uniquely associated with a respective sample in the plurality of samples, wherein the target-index sequences are pooled for sequencing during a sequencing run, and wherein the target sequences are sequenced during target sequencing cycles of the sequencing run and the index sequences are sequenced during index sequencing cycles of the sequencing run, the method including:
accessing target images generated for the target sequences during the target sequencing cycles, wherein the target images depict intensity emissions generated as a result of nucleotide incorporation in the target sequences;
preprocessing the target images using a normalization function that produces a normalized version of a target image from a current target sequencing cycle based on (i) intensity values of target images from one or more preceding target sequencing cycles, (ii) intensity values of target images from one or more succeeding target sequencing cycles, and (iii) intensity values of target images from the current target sequencing cycle;
accessing index images generated for the index sequences during the index sequencing cycles, wherein the index images depict intensity emissions generated as a result of nucleotide incorporation in the index sequences;
preprocessing the index images using the normalization function that produces a normalized version of an index image from a current index sequencing cycle based on (i) intensity values of index images from one or more preceding index sequencing cycles, (ii) intensity values of index images from one or more succeeding index sequencing cycles, and (iii) intensity values of index images from the current index sequencing cycle;
processing normalized versions of the target images through a neural network-based base caller and generating a base call for each of the target sequencing cycles, thereby producing target reads for the target sequences;
processing normalized versions of the index images through the neural network-based base caller and generating a base call for each of the index sequencing cycles, thereby producing index reads for the index sequences; and
classifying each target read of a target sequence as belonging to a particular sample in the plurality of samples based on a corresponding index read of an index sequence that is coupled to the target sequence.

20. The artificial intelligence-based method of claim 19, wherein the normalization function calculates
a lower percentile of (i) the intensity values of the target images from the one or more preceding target sequencing cycles, (ii) the intensity values of the target images from the one or more succeeding target sequencing cycles, and (iii) the intensity values of the target images from the current target sequencing cycle, and
an upper percentile of (i) the intensity values of the target images from the one or more preceding target sequencing cycles, (ii) the intensity values of the target images from the one or more succeeding target sequencing cycles, and (iii) the intensity values of the target images from the current target sequencing cycle, such that,
in the normalized version of the target image,
a first percentage of normalized intensity values are below the lower percentile,
a second percentage of the normalized intensity values are above the upper percentile, and
a third percentage of the normalized intensity values are between the lower and upper percentiles.
